(19) United States
(12) Patent Application Publication
(10) Pub. No.: US 2022/0041667 A1
(43) Pub. Date: Feb. 10, 2022

(54) TARGETING OF MAKAP-PDE4D3 COMPLEXES IN NEURODEGENERATIVE DISEASE

(71) Applicants: The Board of Trustees of the Leland Stanford Junior University, Stanford, CA (US); The University of Miami, Miami, FL (US); The United States Government As Represented By The Department of Veterans Affairs, Washington, DC (US)

(72) Inventors: Michael S. Kapiloff, Los Altos, CA (US); Jeffrey L. Goldberg, Menlo Park, CA (US)

(21) Appl. No.: 17/290,174

(22) PCT Filed: Nov. 13, 2019
(86) PCT No.: PCT/US2019/061211

§ 371 Date: Apr. 29, 2021

Related U.S. Application Data
(60) Provisional application No. 62/767,307, filed on Nov. 14, 2018.

(51) Int. Cl. C07K 14/47; C12N 15/86

(52) U.S. Cl. CPC C07K 14/4702; A61K 38/00; C12N 15/86

(57) ABSTRACT

Nervous system trauma and neurodegeneration including in optic neuropathies are treated by administration of an effective dose of a PDE4D3 displacing agent to promote neurite extension, neuroprotection and recovery. In some embodiments the neurons are optic neurons, including without limitation retinal ganglion cells (RGCs). A cAMP signaling compartment restricted by mAKAPα-anchored PDE4D3 directly regulates neuronal phenotype, and can be molecularly manipulated with therapeutic effect.

### FEDERALLY SPONSORED RESEARCH AND DEVELOPMENT

This invention was made with Government support under contract EY022129, EY026766, EY022129, EY026877, and EY025915 awarded by the National Institutes of Health. The Government has certain rights in the invention.

### BACKGROUND

Intracellular signal transduction is conveyed by second messengers that can act either by diffusion throughout the cell or within discrete functional compartments to modulate diverse target effectors. The morphology of the neuron lends itself to compartmentalized signaling, given the extraordinarily large distances that often exist between axons, dendrites, and the soma, as well as due to the physical constraints upon diffusion conferred by the geometry of structures such as dendritic spines. Despite being a water soluble, inherently diffusible second messenger, cAMP is subject to extensive compartmentation, especially with regards to the regulation of its canonical effector PKA.

Localized to different cellular compartments by binding to A-kinase anchoring proteins (AKAPs), PKA is often found associated with specific adenylyl cyclase and phosphodiesterase (PDE) isoenzymes, such that cAMP fluxes can be locally modulated to regulate individual cellular processes. The functional significance of AKAP-mediated compartmentalization to neuronal function has been studied primarily in terms of synaptic transmission, most prominently cAMP signaling orchestrated by the post-synaptic scaffold protein AKAP79/150 that has an important role in synaptic plasticity and learning and memory. Little is known, however, whether similar microdomains play a role in other cAMP-dependent neuronal functions, including the development of nervous system connectivity, neuronal metabolism, and neuroprotection.

mAKAP (AKAP6) is a modular scaffold protein localized to the nuclear envelope in hippocampal neurons and retinal ganglion cells (RGCs), as well as cardiac and skeletal myocytes. mAKAP was initially identified as a PKA scaffold. Phosphodiesterases (PDEs) are key to the maintenance of cAMP compartmentalization and the prevention of excess cAMP signaling. Type 4 PDE activity is known to suppress axonal regeneration after optic nerve injury. The PDE4D3 isoform is specifically associated with mAKAP through the direct binding by a discrete domain within mAKAP of the N-terminal 4D3 peptide in the phosphodiesterase.

mAKAP orchestrates large multimolecular signalosomes (>25 binding partners identified) that transduce not only cAMP, but also calcium, phospholipid, mitogen-activated protein kinase and hypoxic signaling. By coordinating crosstalk between multiple signaling pathways, mAKAPβ is important in the heart for hypertrophic gene expression and pathological remodeling and in skeletal muscle for myogenic differentiation. mAKAPα expression is required for neurotrophic factor-dependent RGC survival and neurite growth in vitro. In addition, mAKAPα expression in vivo is required for the pro-survival effects of exogenous neurotrophic- and cAMP analogs in mice subjected to optic nerve crush, a model for traumatic optic neuropathy and glaucoma in which RGCs die via retrograde degeneration following damage to their axons. See, for example, Wang, et al. EBioMedicine 2, 1880-1887, (2015).

While mAKAPβ signaling mechanisms are relatively well studied in myocytes, the mechanisms by which mAKAPα signalosomes contribute to neuroprotection and neurite extension remain unknown, including whether cAMP at mAKAPα signalosomes is relevant to these processes. The ability to regulate and enhance neuronal survival and recovery os of great clinical interest and is provided by the methods described herein.

### SUMMARY

Methods and compositions are provided for treatment of damage to, or degenerative diseases of, the nervous system, including neurons and glial cells in the brain, spinal cord and visual system including the retina and optic nerves. Such treatments can be applied to nervous system cells after trauma, or in neurodegenerative diseases including without limitation glaucoma, traumatic optic neuropathy, ischemic optic neuropathy, retinal or macular degeneration whether age-related or inherited, Alzheimer's disease, stroke, etc., to promote neurite extension and neuroprotection and recovery from injury. In some embodiments affected neurons are visual system neurons, including without limitation retinal ganglion cells (RGCs). It is shown herein that a cAMP signaling compartment restricted by mAKAPα-anchored PDE4D3 directly regulates neuronal phenotype, and can be molecularly manipulated with therapeutic effect.

A PDE4D3 displacing agent is provided for manipulating the cAMP signaling compartment of neurons and enhancing neuroprotection and survival. In some embodiments the displacing agent is a peptide. In some embodiments the peptide comprises or consists of a fragment of the PDE4D3 N-terminal sequence. In some embodiments the peptide comprises or consists of the sequence (SEQ ID NO:1) MMHVNNFPFRRHXWICFDVD, where X is any amino acid. In some embodiments X is S. In preferred embodiments, X is E. In some embodiments the peptide of SEQ ID NO:1 is fused to a protein other than PDE4D3, e.g. a matrix protein, a detectable marker, etc.

In some embodiments a PDE4D3 displacing agent is a peptide, which is administered in the form of a nucleic acid encoding the peptide, where the nucleic acid is operably joined to a promoter sequence that is active in the neuronal cell. In other embodiments the PDE4D3 displacing agent disrupts expression of PDE4D, e.g. by providing a sequence comprising PDE4D3-specific siRNA or shRNA. In some embodiments the nucleic acid is provided in a vector. In some embodiments the vector is a plasmid. In some embodiments the vector is a virus. In some embodiments the virus is an adenovirus or an adeno-associated virus (AAV). In some embodiments the virus is administered systemically. In other embodiments the virus is administered locally, e.g. by topical application, intravitreal injection, etc.

In some embodiments a PDE4D3 displacing agent is a peptide, which is administered in the form of a cell-permeable peptide, e.g. fused to a transporter domain. In other embodiments the peptide is administered locally, e.g. by topical application, intravitreal injection, etc.

In some embodiments, administration of a PDE4D3 displacing agent is performed in combination with activation or administration of a neurotrophic factor, or visual or electrical stimulation, where the activity of the neurotrophic factor or visual or electrical stimulation is potentiated by administration of the PDE4D3 displacing agent. In some embodiments the neurotrophic factor is one or more of brain-derived neurotrophic factor (BDNF), ciliary neurotrophic factor (CNTF), glial cell line-derived neurotrophic factor (GDNF), neurotrophin-4, sciatic nerve (ScN)-derived factor, etc.

Methods are provided for protecting or treating an individual suffering from adverse effects of optic neuropathy by administering an effective dose of a PDE4D3 displacing agent, including administration by localized delivery to the optic nerve. These findings have broad implications for a variety of clinical conditions, including traumatic optic nerve injury, glaucoma, ischemic nerve injury, Alzheimer's disease, stroke, and other conditions.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1E. Perinuclear localization of mAKAPα at nesprin-1α is required for primary hippocampal neuron neurite outgrowth. FIG. 1A Structure of mAKAPα and expressed proteins. The three spectrin repeats (SR) required for nuclear envelope targeting are indicated. Binding sites are shown for those mAKAP binding partners for which there is evidence of direct binding: PDK1, 3-phosphoinositide-dependent kinase-1, AC5, adenylyl cyclase 5, MEF2, PLCε, phospholipase Cε, nesprin-1α, RyR2, ryanodine receptor, CaN, calcineurin, PDE4D3, phosphodiesterase 4D3, RSK3, p90 ribosomal S6 kinase 3, PKA, protein kinase A, and PP2A, protein phosphatase 2A. FIG. 1B Hippocampal neurons stained with α-nesprin (green) and α-MAP2 antibodies (red) and DAPI nuclear stain (blue) with grayscale single channel images. FIG. 1C Displacement of mAKAP by mAKAP-SR-GFP. Neurons expressing a mAKAP-DsRed fusion protein (red in composite and shown separately as grayscale image) and either GFP (green) or mAKAP-SR-GFP (green) and stained with DAPI (blue). Scale bar—10 μm and n=3 for both FIGS. 1B-1D. Neurons expressing mAKAP-SR-GFP or GFP control were cultured in defined media in the presence or absence of 40 mM KCl for 2 days. Grayscale images of GFP fluorescence are shown. Scale bar—100 μm. FIG. 1E Quantification of neurite outgrowth. The length of the longest neurite was measured. Colors represent paired data for 4 independent experiments.

FIG. 2A-2E. Characterization of a new perinuclear PKA FRET sensors. FIG. 2A AKAR4 is a cerulean-cpVenus FRET sensor that exhibits increased signal upon phosphorylation of the PKA peptide substrate. PN-AKAR4 is an AKAR4-nesprin-1α fusion protein. FIG. 2B Grayscale CFP images of Cos-7 cells expressing AKAR4 or PN-AKAR4. Scale bar—10 μm. FIG. 2C Average normalized FRET ratio signal+/−s.e.m. (R/R₀) following stimulation with 10 μM FSK and 100 μM IBMX and then inhibition with 10 μM H89. FIG. 2D-2E Cos-7 cells expressing sensor and either mAKAPα WT or PKA binding mutant (mAKAPΔPKA) were stimulated with 10 μM FSK for 2 min (bar on graph). Average tracings (R/R₀+/−s.e.m.) and the peak amplitude and half-time of signal decay (t_{1/2}) for individual tracings are shown; red bars indicate mean.

FIG. 3A-3E. PN-AKAR4 is an mAKAPα-dependent PKA sensor when expressed in hippocampal neurons. FIG. 3A Grayscale CFP images of PN-AKAR4 and AKAR4 sensors in neurons. Scale bar—100 μm. FIG. 3B Co-localization of mAKAPα-DsRed and PN-AKAR4. Scale bar—10 μm. FIG. 3C-3E. Neurons were infected with adenovirus for PN-AKAR4 or AKAR4 and for mAKAP or control shRNA and stimulated with 10 μM FSK for 2 min (horizontal bars). Average tracings (R/R₀+/−s.e.m.) and the peak amplitude and half-time of signal decay (t_{1/2}) for individual tracings are shown; red bars indicate mean.

FIG. 4A-4E. Elevated perinuclear cAMP is sufficient to promote neurite outgrowth. FIG. 4A In “mCherry-AC-nesprin,” mCherry and the constitutively active catalytic domain of ADCY10 are fused to the N-terminus of full-length nesprin-1α. FIG. 4B-4C Baseline FRET ratio (R₀=net FRET÷Donor) for PN-AKAR4 (n=14, 16) and AKAR4 (n=9-19) was measured using hippocampal neurons expressing mCherry-AC-nesprin or control mCherry-nesprin; red bars indicate mean. FIG. 4D Hippocampal neurons expressing GFP and either mCherry-nesprin control or mCherry-AC-nesprin were cultured in defined media in the absence or presence of KCl for 2 days. Grayscale images of GFP fluorescence are shown. Scale bar—100 μm. FIG. 4E The length of the longest neurite was measured. n=4-8 independent neuronal cultures.

FIG. 5A-5I. Perinuclear cAMP is required for neurite outgrowth in hippocampal neurons. FIG. 5A In “mCherry-PDE-nesprin,” mCherry and a constitutively active catalytic domain of PDE4D are fused to the N-terminus of full-length nesprin-1α. FIG. 5B, 5D, 5F. Baseline FRET ratio (R₀=net FRET÷Donor) was measured using neurons expressing PN-AKAR4 or AKAR4 and mCherry-PDE-nesprin or control mCherry-nesprin; red bars indicate mean. FIG. 5C, 5E, 5G. FRET tracings were obtained following stimulation with 10 μM FSK for 2 min (horizontal bars). Average tracings (R/R₀+/−s.e.m.) and the peak amplitude and half-time of signal decay (t_{1/2}) for individual tracings are shown. FIG. 5AH Neurons expressing GFP and either mCherry-nesprin control or mCherry-PDE-nesprin were cultured in defined media in the absence or presence of KCl for 2 days. Grayscale images of GFP fluorescence are shown. Scale bar—100 μm. FIG. 5I. The length of the longest neurite was measured. n=4-10 independent neuronal cultures.

FIG. 6A-6B. Pharmacological induction of neurite outgrowth. FIG. 6A Hippocampal neurons transfected with a GFP expression plasmid were treated with 40 mM KCl, 10 μM FSK, 100 μM IBMX, 20 μM milrinone or 10 μM rolipram for 2 days. Grayscale images of GFP fluorescence are shown. Scale bar 100 μm. FIG. 6B Mean length of the longest neurite. Colors represent paired data for 4 independent experiments.

FIG. 7A-7K. Displacement of PDE4D3 from mAKAPα increases perinuclear cAMP and promotes hippocampal and RGC neurite extension. FIG. 7A 4D3(E)-mCherry includes the PDE4D3 isoform-specific N-terminal peptide with a Ser13Glu substitution in fusion to mCherry. FIG. 7B, 7D, 7F. Baseline FRET ratio (R₀=net FRET÷Donor) was measured using hippocampal neurons expressing PN-AKAR4 or AKAR4 and 4D3(E)-mCherry or control mCherry; red bars indicate mean. FIG. 7C, 7E, 7G. Tracings were obtained following stimulation for 2 min with 10 μM forskolin (bar on graphs showing average R/R₀+/−s.e.m.). Peak amplitude and half-time of signal decay (t_{1/2}) for individual tracings are shown on right. FIG. 7H, 7I. Grayscale images of mCherry fluorescence for hippocampal neurons transfected with mCherry or 4D3(E)-mCherry expression plasmids and cultured for 2 days in defined media. Scale bar—100 μm. FIG. 7I. Mean length of the longest neurite are shown for 4 independent experiments (different colors). FIG. 7J, 7K. Same as in FIG. 7I except using RGCs.

FIG. 8A-8F. PDE4D3 anchoring disruption increases RGC survival after optic nerve crush. FIG. 8A 4D3(E)-mCherry was expressed in vivo using the gene therapy vector AAV2.4D3(E). FIG. 8B Retinas isolated two weeks after optic nerve crush were stained for the RGC marker RBPMS (shown in grayscale). Scale—100 μm. c. Quantification of RBPMS-stained cells showing increased RGC survival after AAV2.4D3(E) injection. n=5,6 mice. FIG. 8D, 8E. same as in FIG. 8B, 8C except performed by a different investigator. n=4,5 mice. FIG. 8F Model for regulation of perinuclear cAMP by mAKAPα signalosomes. mAKAPα binds the cAMP-specific, PKA-activated phosphodiesterase PDE4D3 that will oppose local PKA signaling in response to cAMP. The 4D3(E) peptide will displace PDE4D3 from mAKAPα potentiating local PKA signaling that promotes neuroprotection and neurite extension.

### DETAILED DESCRIPTION OF THE EMBODIMENTS

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

An effective dose of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials. The displacing agent is administered at a dosage, alone or in combination with other agents, that enhances neuron recovery while minimizing any side-effects. The effectiveness of recovery may be assessed, for example, by monitoring function of the neuron, e.g. maintenance or recovery of vision in glaucoma patients, such as at least about 5% recovery, at least about 10% recovery, at least about 20%, at least about 30%, at least about 40%, at least about 50%, at least about 75%, at least about 85%, at least about 95% or more, e.g. assessing by conventional measures of vision or retinal or optic nerve structure in the case of glaucoma or other optic neuropathies, or function or structure of the brain or spinal cord where used in diseases or traumas or strokes that affect those tissues and are treated by the methods herein. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

By “neurological” or “cognitive” function as used herein, it is meant the patient's ability to think, function, etc. In conditions where there is axon loss and regrowth, there may be recovery of motor and/or sensory abilities.

By “neurodegenerative disease, disorder, or condition” is meant a disease, disorder, or condition (including a neuropathy) associated with degeneration or dysfunction of neurons or other neural cells throughout the nervous system, including but not limited to those in the retina such as retinal ganglion cells or photoreceptor cells. A neurodegenerative disease, disorder, or condition can be any disease, disorder, or condition in which decreased function or dysfunction of neurons, or loss or neurons or other neural cells, can occur.

As used herein, a “neuron or portion thereof” can consist of or be a portion of a neuron, for example a retinal ganglion cell, and the like. More particularly, the term “neuron” as used herein denotes nervous system cells that include a central cell body or soma, and two types of extensions or projections: dendrites, by which, in general, the majority of neuronal signals are conveyed to the cell body; and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons).

In some embodiments, the neuron or portion thereof can be present in a subject, such as a human subject. The subject can, for example, have or be at risk of developing a disease, disorder, or condition of the nervous system, an injury to the nervous system, such as, for example, an injury caused by physical, mechanical, or chemical trauma; ocular-related neurodegeneration; and the like. By “neurodegenerative disease, disorder, or condition” is meant a disease, disorder, or condition (including a neuropathy) associated with degeneration or dysfunction of neurons or other neural cells, such as retinal ganglion cells or photoreceptor cells. Examples of ocular-related neurodegeneration include, but are not limited to, glaucoma, retinitis pigmentosa, age-related macular degeneration (AMD), photoreceptor degeneration associated with wet or dry AMD, other retinal degeneration, optic nerve drusen, ischemic or traumatic optic neuropathy, and optic neuritis.

Examples of injuries to the nervous system caused by physical, mechanical, or chemical trauma include, but are not limited to, nerve damage caused by ischemia, exposure to toxic compounds, heavy metals (e.g., lead, arsenic, and mercury), industrial solvents, drugs, chemotherapeutic agents, dapsone, HIV medications (e.g., zidovudine, didanosine, stavudine, zalcitabine, ritonavir, and amprenavir), cholesterol lowering drugs (e.g., lovastatin, indapamide, and gemfibrozil), heart or blood pressure medications (e.g., amiodarone, hydralazine, perhexiline), and metronidazole. More particularly, traumatic injury or other damage to neuronal cells (e.g., trauma due to accident, blunt-force injury, gunshot injury, spinal cord injury, ischemic conditions of the nervous system such as stroke, cell damage due to aging or oxidative stress, and the like) also is intended to be included within the language “neurodegenerative disease, disorder, or condition.” In such embodiments, the presently disclosed methods can be used to treat neuronal damage due to traumatic injury or stroke by preventing death of damaged neuronal cells and/or by promoting or stimulating neurite growth from damaged neuronal cells.

Further examples also include burn, wound, surgery, accidents, ischemia, prolonged exposure to cold temperature, stroke, intracranial hemorrhage, and cerebral hemorrhage. More particularly, traumatic injury or other damage to neuronal cells, e.g., trauma due to accident, blunt-force injury, gunshot injury, spinal cord injury, ischemic conditions of the nervous system such as stroke, cell damage due to aging or oxidative stress, and the like is also included within the language “neurodegenerative disease, disorder, or condition.” In such embodiments, the presently disclosed methods can be used to treat neuronal damage due to traumatic injury or stroke by preventing death of damaged neuronal cells and/or by promoting or stimulating neurite growth from damaged neuronal cells.

In some embodiments, the subject is suffering from or susceptible to a neurodegenerative disease, disorder, or condition, such as glaucoma, e.g., a subject diagnosed as suffering from or susceptible to a neurodegenerative disease, disorder, or condition. In other embodiments, the subject has been identified (e.g., diagnosed) as suffering from or susceptible to a neurodegenerative disease, disorder, or condition (including traumatic injury) in which neuronal cell loss is implicated, or in which damage to neurites is involved, and for which treatment or prophylaxis is desired.

In some embodiments, the presently disclosed methods include preventing or inhibiting neuron or axon degeneration. Preventing axon or neuron degeneration includes decreasing or inhibiting axon or neuron degeneration, which may be characterized by complete or partial inhibition of neuron or axon degeneration. Such prevention or inhibition can be assessed, for example, by analysis of neurological function. Further, the phrases “preventing neuron degeneration” and “inhibiting neuron degeneration” include such inhibition with respect to the entire neuron or a portion thereof, such as the neuron cell body, axons, and dendrites.

Administration of a PDE4D3 displacing agent, alone or in combination with activation or administration of a neurotrophic factor, is useful for treatment of injuries to the retinal ganglia that are caused by mechanical forces, such as a blow to the head or spine, and which, in the absence of treatment, result in neuronal death, or severing of axons. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from ischemia, constriction or compression of ganglia by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

Of particular interest is administration of a PDE4D3 displacing agent, alone or in combination with activation or administration of a neurotrophic factor, for treatment of glaucoma. Glaucomas are a group of eye disorders characterized by progressive optic nerve damage in which an important part is a relative increase in intraocular pressure (IOP). Glaucoma is the 2nd most common cause of blindness worldwide. Glaucoma can occur at any age but is 6 times more common among people >60 yr.

Glaucomas are categorized as open-angle glaucoma, closed-angle glaucoma. Glaucomas are further subdivided into primary (cause of outflow resistance or angle closure is unknown) and secondary (outflow resistance results from a known disorder).

Glaucoma patients with characteristic optic nerve and corresponding visual field changes should be treated regardless of 10P measurement, for example by administration of an effective dose of a PDE4D3 displacing agent, alone or in combination activation or administration of a neurotrophic factor and/or visual or electrical stimulation, where the activity of the neurotrophic factor is potentiated by administration of the PDE4D3 displacing agent.

Non-limiting examples of different types of glaucoma that can be prevented or treated according to the presently disclosed subject matter include primary glaucoma (also known as primary open-angle glaucoma, chronic open-angle glaucoma, chronic simple glaucoma, and glaucoma simplex), low-tension glaucoma, primary angle-closure glaucoma (also known as primary closed-angle glaucoma, narrow-angle glaucoma, pupil-block glaucoma, and acute congestive glaucoma), acute angle-closure glaucoma, chronic angle-closure glaucoma, intermittent angle-closure glaucoma, chronic open-angle closure glaucoma, pigmentary glaucoma, exfoliation glaucoma (also known as pseudoexfoliative glaucoma or glaucoma capsulare), developmental glaucoma (e.g., primary congenital glaucoma and infantile glaucoma), secondary glaucoma (e.g., inflammatory glaucoma (e.g., uveitis and Fuchs heterochromic iridocyclitis)), phacogenic glaucoma (e.g., angle-closure glaucoma with mature cataract, phacoanaphylactic glaucoma secondary to rupture of lens capsule, phacolytic glaucoma due to phacotoxic meshwork blockage, and subluxation of lens), glaucoma secondary to intraocular hemorrhage (e.g., hyphema and hemolytic glaucoma, also known as erythroclastic glaucoma), traumatic glaucoma (e.g., angle recession glaucoma, traumatic recession on anterior chamber angle, postsurgical glaucoma, aphakic pupillary block, and ciliary block glaucoma), neovascular glaucoma, drug-induced glaucoma (e.g., corticosteroid induced glaucoma and alpha-chymotrypsin glaucoma), toxic glaucoma, and glaucoma associated with intraocular tumors, retinal detachments, severe chemical burns of the eye, and iris atrophy.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% decrease in cell loss or loss of function relative to cell survival or cell function measured in absence of the PDE4D3 displacing agent. Treatment may result in at least about a 10%, 15%, 20%, 25%, 30%, 35%, 40%, 45%, 50%, 55%, 60%, 65%, 70%, 75%, 80%, 85%, 90%, 95%, or even 100% decrease in symptoms of a disease, disorder, or condition of the nervous system, compared to a subject that is not treated with a PDE4D3 displacing agent.

Phosphodiesterase 4D (PDE4D) is a class IV cAMP-specific PDE. The PDE4D gene is complex, spanning just under 1 Mb with 17 exons and encoding at least 9 different variants encoding functional proteins, of which PDE4D3 is one. PDE4D3 shows cAMP PDE activity, which was inhibited by several cyclic nucleotide PDE inhibitors. A cAMP-responsive signaling complex maintained by the muscle-specific A-kinase anchoring protein (mAKAP, also known as AKAP6) includes PKA, PDE4D3, and EPAC1. These intermolecular interactions facilitate the dissemination of distinct cAMP signals through each effector protein. Anchored PKA stimulates PDE4D3 to reduce local cAMP concentrations, whereas an AKAP6-associated ERK5 kinase module suppresses PDE4D3. PDE4D3 also functions as an adaptor protein that recruits EPAC1, an exchange factor for the small GTPase RAP1, to enable cAMP-dependent attenuation of ERK5. Pharmacologic and molecular manipulations of the AKAP6 complex show that anchored ERK5 can induce cardiomyocyte hypertrophy.

The amino acid and genetic sequence of human cAMP-specific 3′,5′-cyclic phosphodiesterase 4D isoform PDE4D3 may be accessed, for example, at Genbank NP_006194. See, for example, Nemoz et al. (1996) FEBS Lett. 384 (1), 97-102; Robertson et al. (1994) Genomics 23 (1), 42-50; Swinnen et al. (1989) Proc. Natl. Acad. Sci. U.S.A. 86 (21), 8197-8201. Isoform PDE4D3 (also known as isoform 2) is shorter and has a distinct N-terminus, compared to isoform PDE4D4. The human protein is 673 amino acids in length, with the sequence as follows (SEQ ID NO:2):

| | |
|---|---|
| 1 | MMHVNNFPFR RHSWICFDVD NGTSAGRSPL DPMTSPGSGL |
| | ILQANFVHSQ RRESFLYRSD |
| 61 | SDYDLSPKSM SRNSSIASDI HGDDLIVTPF AQVLASLRTV |
| | RNNFAALTNL QDRAPSKRSP |
| 121 | MCNQPSINKA TITEEAYQKL ASETLEELDW CLDQLETLQT |
| | RHSVSEMASN KFKRMLNREL |
| 181 | THLSEMSRSG NQVSEFISNT FLDKQHEVEI PSPTQKEKEK |
| | KKRPMSQISG VKKLMHSSSL |
| 241 | TNSSIPRFGV KTEQEDVLAK ELEDVNKWGL HVFRIAELSG |
| | NRPLTVIMHT IFQERDLLKT |
| 301 | FKIPVDTLIT YLMTLEDHYH ADVAYHNNIH AADVVQSTHV |
| | LLSTPALEAV FTDLEILAAI |
| 361 | FASAIHDVDH PGVSNQFLIN TNSELALMYN DSSVLENHHL |
| | AVGFKLLQEE NCDIFQNLTK |
| 421 | KQRQSLRKMV IDIVLATDMS KHMNLLADLK TMVETKKVTS |
| | SGVLLLDNYS DRIQVLQNMV |
| 481 | HCADLSNPTK PLQLYRQWTD RIMEEFFRQG DRERERGMEI |
| | SPMCDKHNAS VEKSQVGFID |
| 541 | YIVHPLWETW ADLVHPDAQD ILDTLEDNRE WYQSTIPQSP |
| | SPAPDDPEEG RQGQTEKFQF |
| 601 | ELTLEEDGES DTEKDSGSQV EEDTSCSDSK TLCTQDSEST |
| | EIPLDEQVEE EAVGEEEESQ |
| 661 | PEACVIDDRS PDT |

A PDE4D3 displacing agent, as used herein, refers to an agent, e.g. a peptide, a nucleic acid, etc., that interferes with the binding of PDE4D3 and mAKAP (either α neural or β muscle mAKAP isoform), causing the displacement of PDE4D3, and thereby increasing cAMP-signaling in the specific compartment associated with mAKAP. Manipulating the cAMP signaling compartment of neurons enhances neuroprotection and survival, and can potentiate the effects of neurotrophic agents and growth factors.

In some embodiments the displacing agent is a peptide, for example a peptide that competes with PDE4D3 for binding to mAKAP. In some embodiments the peptide comprises or consists of a fragment of the PDE4D3 N-terminal sequence. The N-terminal sequence generally corresponds to the amino acid sequence of SEQ ID NO:2, comprising or consisting of at least residues 1-20, although the N-terminal sequence may be optionally extended to include, for example, residues 1-22, 1-25, 1-27, 1-30, 1-35, 1-40, etc. Alternatively the N-terminal sequence may be truncated by 1, 2, 3 or more residues, for example comprising residues 2-20, 3-20, 4-20, etc.

In some embodiments the peptide comprises or consists of the sequence (SEQ ID NO:1) MMHVNNFPFRRHXWICFDVD, where X is any amino acid. In some embodiments X is S. In a preferred embodiments, X is E. In some embodiments the peptide of SEQ ID NO:1 is fused to a protein other than PDE4D3, e.g. a matrix protein, a detectable marker, etc.

In some embodiments a PDE4D3 displacing agent is a peptide administered in the form of a cell-permeable peptide, e.g. fused to a transporter domain. In other embodiments the peptide is administered locally, e.g. by topical application, intravitreal injection, etc.

A number of transporter (permeant) domains are known in the art and may be used in the present invention, including peptides, peptidomimetics, and non-peptide carriers. In one embodiment, the permeant peptide is derived from the third alpha helix of Drosophila melanogaster transcription factor Antennapaedia, referred to as penetratin, which comprises the amino acid sequence RQIKIWFQNRRMKWKK. In another embodiment, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

The sequence of a peptide displacing agent may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In other embodiments a PDE4D3 displacing agent is a peptide produced in the targeted cell, but administered in the form of a nucleic acid encoding the peptide, where the nucleic acid is operably joined to a promoter sequence that is active in the neuronal cell. In other embodiments the PDE4D3 displacing agent disrupts expression of PDE4D, e.g. by providing a sequence comprising PDE4D3-specific siRNA or shRNA. In some embodiments the nucleic acid is provided in a vector. In some embodiments the vector is a plasmid. In some embodiments the vector is a virus. In some embodiments the virus is an adenovirus or an adeno-associated virus (AAV). In some embodiments the virus is administered systemically. In other embodiments the virus is administered locally, e.g. by topical application, intravitreal injection, etc.

In such methods, sequences encoding a PDE4D3 displacing agent or PDE4D3-specific siRNA or shRNA are introduced into the nervous system, including the optic nerve, and expressed, as a means of providing activity to the targeted cells. In one approach, genetic “vectors” are injected directly into one or more regions in the nervous, to genetically alter cells. It should be noted that the terms “transfect” and “transform” are used interchangeably herein. Both terms refer to a process which introduces a foreign gene (also called an “exogenous” gene) into one or more preexisting cells, in a manner which causes the foreign gene(s) to be expressed to form corresponding polypeptides. This has been achieved by directly injecting a genetic vector, to introduce foreign genes into neurons “in situ” (i.e., neurons which remain in their normal position, inside a patient's brain or spinal cord, throughout the entire genetic transfection or transformation procedure).

Useful vectors include viral vectors, which make use of the lipid envelope or surface shell (also known as the capsid) of a virus. These vectors emulate and use a virus's natural ability to (i) bind to one or more particular surface proteins on certain types of cells, and then (ii) inject the virus's DNA or RNA into the cell. In this manner, viral vectors can deliver and transport a genetically engineered strand of DNA or RNA through the outer membranes of target cells, and into the cells cytoplasm. Gene transfers into CNS neurons have been reported using such vectors derived from herpes simplex viruses (e.g., European Patent 453242, Breakfield et al 1996), adenoviruses (La Salle et al 1993), and adeno-associated viruses (Kaplitt et al 1997).

Vectors typically contain the transcriptional regulatory elements necessary for expression of the desired gene, and may include an origin of replication, selectable markers and the like, as known in the art. A vector may comprise selected agents that can aid entry of the gene construct into target cells. Several commonly-used agents include cationic lipids, positively charged molecules, and/or ligands that bind to receptors expressed on the surface of the target cell. Examples of positively-charged transfection agents include polylysine, polyethylenimine (PEI), and various cationic lipids. The basic procedures for preparing genetic vectors using cationic agents are similar. A solution of the cationic agent (polylysine, PEI, or a cationic lipid preparation) is added to an aqueous solution containing DNA (negatively charged) in an appropriate ratio. The positive and negatively charged components will attract each other, associate, condense, and form molecular complexes. If prepared in the appropriate ratio, the resulting complexes will have some positive charge, which will aid attachment and entry into the negatively charged surface of the target cell. The use of liposomes to deliver foreign genes into sensory neurons is described in various articles such as Sahenk et al 1993. The use of PEI, polylysine, and other cationic agents is described in articles such as Li et al 2000 and Nabel et al 1997.

An alternative strategy for introducing DNA into target cells is to associate the DNA with a molecule that normally enters the cell. Known agents that bind to neuronal receptors and trigger endocytosis, causing them to enter the neurons, include (i) the non-toxic fragment C of tetanus toxin; (ii) various lectins derived from plants, such as barley lectin and wheat germ agglutinin lectin; and, (iii) certain neurotrophic factors (e.g., Barde et al 1991). At least some of these endocytotic agents undergo “retrograde” axonal transport within neuron

A vector of particular interest is the adeno-associated virus (AAV), which is a small, non-pathogenic dependovirus that has not been associated with human disease, and in the absence of co-infection with a helper virus such as adenovirus or herpes simplex virus, AAV is unable to replicate. AAV virions, which are non-enveloped and measure 25 nm in diameter, have a genome of 4.9 kB. The AAV genome, which is single-stranded DNA, consists of three open reading frames (ORFs) flanked by two inverted terminal repeats (ITRs), which are 145 bp palindromic sequences that form elaborate hairpin structures and are essential for viral packaging. The first ORF is rep, which encodes 4 proteins involved in viral replication (Rep40, Rep52, Rep68, and Rep72). The second ORF contains cap, which encodes the three structural proteins that make up the icosahedral AAV capsid (VP1, VP2, and VP3). A third ORF, which exists as a nested alternative reading frame in the cap gene, encodes the assembly-activating protein, which localizes AAV capsid proteins to the nucleolus and participates in the process of capsid assembly. AAV has proven to be a safe and efficient vehicle for delivering therapeutic DNA to numerous tissue targets, in particular retinal neurons, and numerous studies have shown the potential of AAV-mediated delivery of genetic material for the treatment of inherited forms of retinal degeneration.

Gene delivery vehicles or vectors based on AAV offer many advantages over other viruses as a vector for the retina. AAV vectors have the ability to infect quiescent cells and give rise to long-term expression of transgenes, and various serotypes exhibit tropisms for different subsets of retinal cells. The delivery efficacy or tropism for different retinal cells implicated in retinal degenerations—including photoreceptors, the retinal pigment epithelium (RPE), Müller glia, and ganglion cells 13 depends on a combination of the capsid and the route of administration, which can be either subretinal to expose virus to photoreceptors and RPE or intravitreal to expose virus primarily to retinal ganglion and Müller cells. AAV2, the best characterized AAV serotype, has been used in clinical trials for Leber's congenital amaurosis type 2 (LCA2), with well-tolerated subretinal administration.

Next generation AAV vectors include, for example, self-complementary vectors (scAAV), whose genomes contain both a sense copy of the transgene and a reverse complement, separated by a linker. These two copies are able to anneal and serve as a double stranded template that can be transcribed without the need for generation of any complementary strand by the host cell. scAAV2, scAAV5 and scAAV8 have been shown to have faster onset of expression in retinal cells, with a similar pattern of expression as the single-stranded vectors. Directed evolution has also been used to develop improved vectors, including viruses capable of better infecting embryonic stem cells, crossing the inner limiting membrane to infect Müller glia from the vitreous, and increased resistance to high affinity antibodies. AAV variants can also be evolved for the ability to infect photoreceptors and RPE from the vitreous.

Promoters useful in an AAV delivered coding sequence may include, for example constitutively active promoters, such as CMV promoters, β-actin promoters, SV-40 promoters such as 4×GRM6-SV40, etc. Commonly used ubiquitous promoters have been immediate-early cytomegalovirus (CMV) enhancer-promoter and the CAG promoter, which combines the CMV enhancer with the chicken β-actin (CBA) promoter. Promoters having more cell-type specific expression patterns may include, without limitation the regulatory region of the gamma-synuclein gene (SNCG), Nefh promoter, Mcp-1 promoter, etc.

As an alternative to viral-based vectors, coding sequences can be introduced by genome editing tools, e.g. the CRISPR)/CRISPR-associated protein 9 (Cas9) system. The Cas9 protein is activated after binding guide RNA (gRNA or sgRNA) by REC1 following a conformational change in the protein. Then, it searches for target DNA stochastically by binding with sequences that matches its PAM sequence and immediately melts the bases of the PAM, paring them with the complementary region on the gRNA. If the matching region and the target region are properly paired, the nuclease domains, RuvC and HNH, will cut the target DNA after the third nucleotide base upstream of the PAM. gRNA or sgRNA are designed to a specific genomic sequence. sgRNAs and Cas9 can be cloned into plasmids and then introduced into mammalian cells by transfection, directing Cas9 to knockout the gene. For long-term expression which will result in stable knockout, Cas9 protein associated with sgRNAs can be pre-packed into lentiviral vectors, and then transduced into target cells. Both the sgRNA and Cas9 are integrated stably into the genome of host cells, and have the ability to pass along to their daughter cells when the cells divide. This will provide permanent expression of shRNA and Cas9.

### Methods of Treatment

Treatment of damage to, or degenerative diseases of, the nervous system, including neurons and glial cells in the brain, spinal cord and visual system including the retina and optic nerves is provided by administration of a PDE4D3 displacing agent. Such treatments can be applied to nervous system cells after trauma, or in neurodegenerative diseases including without limitation glaucoma, traumatic optic neuropathy, ischemic optic neuropathy, retinal or macular degeneration whether age-related or inherited, Alzheimer's disease, stroke, etc., to promote neurite extension and neuroprotection and recovery from injury.

In some embodiments optic neuropathy, including without limitation glaucoma, traumatic optic neuropathy, ischemic optic neuropathy, etc., is treated by administration of a PDE4D3 displacing agent as described herein, to manipulate the cAMP signaling compartment of neurons and enhance neuroprotection and survival of the neuron. In some embodiments the neurons are optic neurons, including without limitation retinal ganglion cells (RGCs).

In some embodiments, administration of a PDE4D3 displacing agent is performed in combination with activation or administration of a neurotrophic factor or visual or electrical stimulation, where the activity of the neurotrophic factor or visual or electrical stimulation is potentiated by administration of the PDE4D3 displacing agent. In some embodiments the neurotrophic factor is one or more of brain-derived neurotrophic factor (BDNF), ciliary neurotrophic factor (CNTF), glial cell line-derived neurotrophic factor (GDNF), neurotrophin-4, sciatic nerve (ScN)-derived factor, etc.

A PDE4D3 displacing agent, including a vector encoding a PDE4D3 displacing agent, can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

In some embodiments the virus is delivered by topical application to the eye, for example, eye drops, intravitreal injection, etc. Intravitreal, subconjunctival, and periocular routes of administration and controlled release formulations of various carriers like nanoparticles, nanoemulsions, microemulsions, dendrimers and microparticles are useful ophthalmic therapeutics. Biodegradable as well as non-biodegradable implants to deliver the agent may be used.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically is within a range of circulating concentrations that include the ED₅₀ with low toxicity. The dosage can vary within this range depending upon the dosage form employed and the route of administration utilized.

Formulations suitable for parenteral or intracranial administration include aqueous and non-aqueous, isotonic sterile injection solutions, which can contain antioxidants, buffers, bacteriostats, and solutes that render the formulation isotonic with the blood vitreous, or cerebrospinal fluid of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Where the therapeutic agents are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

For intracerebral use, the compounds can be administered continuously by infusion into the fluid reservoirs of the CNS, although bolus injection may be acceptable. The displacing agent can be administered into the ventricles of the brain or otherwise introduced into the CNS or spinal fluid. Administration can be performed by use of an indwelling catheter and a continuous administration means such as a pump, or it can be administered by implantation, e.g., intracerebral implantation of a sustained-release vehicle. More specifically, the presently disclosed compounds can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of cholinergic agonists to Alzheimer's disease and of dopamine or dopamine agonists for Parkinson's disease patients.

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. some agents may be taken for extended periods of time on a daily or semi-daily basis, while more selective agents may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

In certain embodiments, the presently disclosed subject matter also includes combination therapies. Depending on the particular disease, disorder, or condition to be treated or prevented, additional therapeutic agents, which are normally administered to treat or prevent that condition, may be administered in combination with the compounds of this disclosure. These additional agents may be administered separately, as part of a multiple dosage regimen. Alternatively, these agents may be part of a single dosage form, mixed together with the PDE4D3 displacing agent.

By “in combination with” is meant the administration of a PDE4D3 displacing agent, or other compounds disclosed herein, with one or more therapeutic agents either simultaneously, sequentially, or a combination thereof. Therefore, a cell or a subject administered a combination of a PDE4D3 displacing agent, can receive one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the PDE4D3 displacing agent and one or more therapeutic agents are administered simultaneously, they can be administered to the cell or administered to the subject as separate pharmaceutical compositions or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered compound is not diminished by the sequential, simultaneous or separate administration of the subsequent compound(s).

For example, in the treatment of glaucoma, other anti-glaucoma medicaments can be used in combination with a PDE4D3 displacing agent, including, but not limited to, beta-blockers, including levobunolol (BETAGAN), timolol (BETIMOL, TIMOPTIC), betaxolol (BETOPTIC) and metipranolol (OPTIPRANOLOL); alpha-agonists, such as apraclonidine (IOPIDINE) and brimonidine (ALPHAGAN); carbonic anhydrase inhibitors, such as acetazolamide, methazolamide, dorzolamide (TRUSOPT) and brinzolamide (AZOPT); prostaglandins or prostaglandin analogs such as latanoprost (XALATAN), bimatoprost (LUMIGAN) and travoprost (TRAVATAN); miotic or cholinergic agents, such as pilocarpine (ISOPTO CARPINE, PILOPINE) and carbachol (ISOPTO CARBACHOL); epinephrine compounds, such as dipivefrin (PROPINE); forskolin; or neuroprotective compounds, such as brimonidine and memantine.

Other combinations include combinations with neurotrophic agents, which include without limitation brain-derived neurotrophic factor (BDNF), ciliary neurotrophic factor (CNTF), glial cell line-derived neurotrophic factor (GDNF), neurotrophin-4, sciatic nerve (ScN)-derived factor, and the like.

In other embodiments, the presently disclosed subject matter includes a combination therapy of administering a PDE4D3 displacing agent in combination with surgery, e.g., surgical relief of intraocular pressure, e.g., via trabeculectomy, laser trabeculoplasty, or drainage implants, and the like.

In the treatment of ALS, for example, the PDE4D3 displacing agent can be administered in combination with Riluzole, minocycline, insulin-like growth factor 1 (IGF-1), and/or methylcobalamin. In the treatment of Parkinson's disease, the PDE4D3 displacing agent can be administered with L-dopa, dopamine agonists, e.g., bromocriptine, pergolide, pramipexole, ropinirole, cabergoline, apomorphine, and lisuride, DOPA decarboxylase inhibitors, and/or MAO-B inhibitors. In the treatment of Alzheimer's disease, the PDE4D3 displacing agent can be administered with acetylcholinesterase inhibitors, e.g., donepezil, galantamine, and rivastigmine and/or NMDA receptor antagonists, e.g., memantine. The combination therapies can involve concurrent or sequential administration, by the same or different routes, as determined to be appropriate by those of skill in the art. The presently disclosed subject matter also includes pharmaceutical compositions and kits including combinations as described herein.

Also provided are a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In some embodiments, the kits comprise one or more containers, including, but not limited to a vial, tube, ampule, bottle and the like, for containing the compound. The one or more containers also can be carried within a suitable carrier, such as a box, carton, tube or the like. Such containers can be made of plastic, glass, laminated paper, metal foil, or other materials suitable for holding medicaments.

In some embodiments, the container can hold a composition that is by itself or when combined with another composition effective for treating or preventing the condition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The presently disclosed kits or pharmaceutical systems also can include associated instructions for using the compounds for treating or preventing a neurodegenerative disease, disorder, or condition, e.g. optic neuritis, including glaucoma. In some embodiments, the instructions include one or more of the following: a description of the active compound; a dosage schedule and administration; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All structural and functional equivalents to the features and method acts of the various embodiments described throughout the disclosure that are known or later come to be known to those of ordinary skill in the art are intended to be encompassed by the features described and claimed herein. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### Example 1

A method of protecting or regenerating neural ganglia cells by locally administering an effective dose of a PDE4D3 anchoring disruptor peptide or a vector encoding such a peptide to a cell

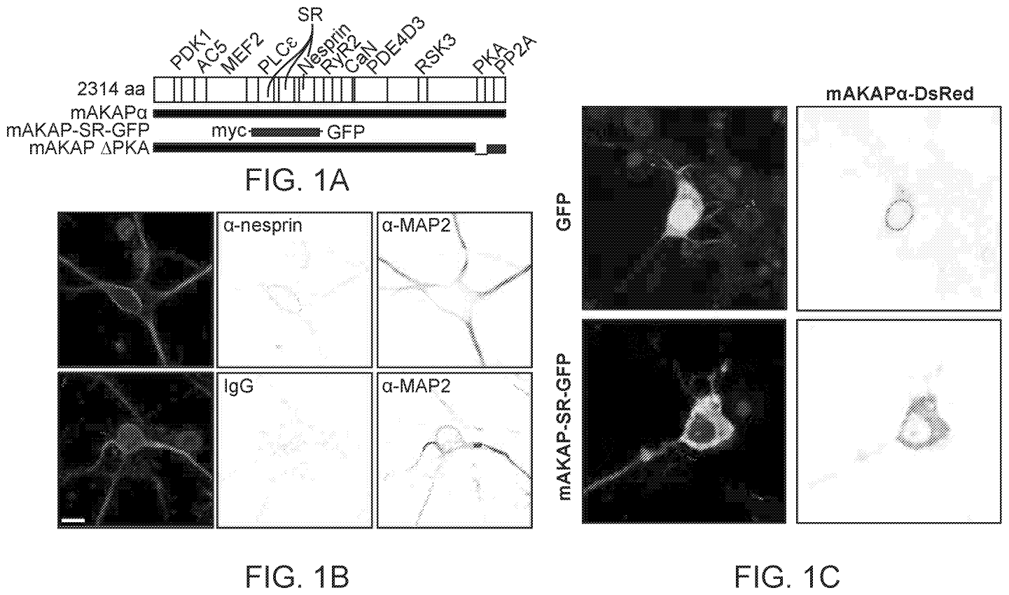

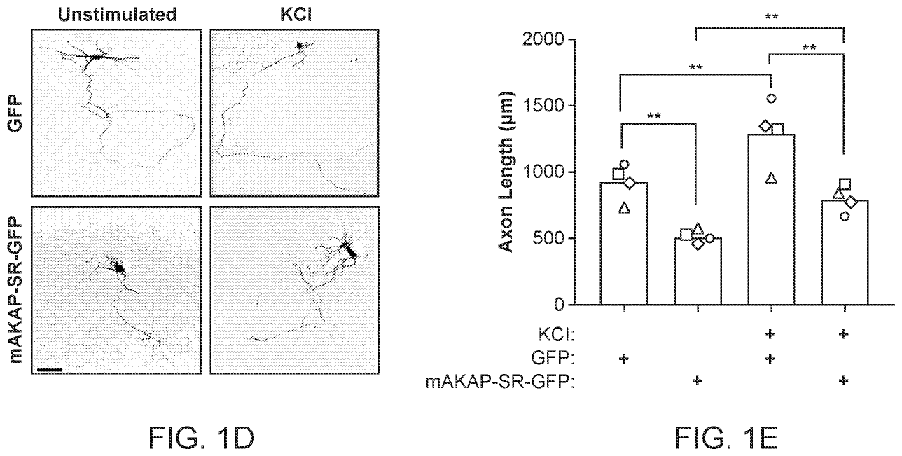

mAKAP (AKAP6, FIG. 1a) is a modular scaffold protein localized to the nuclear envelope in hippocampal neurons and retinal ganglion cells (RGCs), as well as cardiac and skeletal myocytes. mAKAP was initially identified as a PKA scaffold. It was later found to bind both type 2 and type 5 adenylyl cyclase and the cAMP-specific PDE isoform PDE4D3, thereby providing the potential infrastructure for entirely local cAMP regulation. Additional research has revealed that mAKAP orchestrates large multimolecular signalosomes (>25 binding partners identified) that transduce not only cAMP, but also calcium, phospholipid, mitogen-activated protein kinase and hypoxic signaling. mAKAP is expressed as 50 kDa alternatively-spliced a isoform in neurons and the 230 kDa β-isoform in striated myocytes. By coordinating crosstalk between multiple signaling pathways, mAKAPβ is important in the heart for hypertrophic gene expression and pathological remodeling and in skeletal muscle for myogenic differentiation. Recently, we discovered that mAKAPα expression is required for neurotrophic factor-dependent RGC survival and neurite growth in vitro. In addition, mAKAPα expression in vivo is required for the pro-survival effects of exogenous neurotrophic- and cAMP analogs in mice subjected to optic nerve crush, a model for traumatic optic neuropathy and glaucoma in which RGCs die via retrograde degeneration following damage to their axons.

While mAKAPβ signaling mechanisms are relatively well studied in myocytes, the mechanisms by which mAKAPα signalosomes contribute to neuroprotection and neurite extension remain unknown, including whether cAMP at mAKAPα signalosomes is relevant to these processes. Using novel tools to specifically modulate cAMP levels at mAKAPα signalosomes, we now show that increased cAMP in that perinuclear compartment promotes neurite extension in vitro and neuroprotection in vivo. Our results reveal a cAMP signaling compartment restricted by mAKAPα-anchored PDE4D3 that directly regulates neuronal phenotype and that can be molecularly manipulated with potential therapeutic effect.

### Results

mAKAPα anchoring by nesprin-1α is required for neurite extension in vitro. mAKAP is localized to the nuclear envelope via protein-protein interactions, a mechanism of which we were able to take advantage during our studies of mAKAPα signalosome function. Klarsicht/ANC-1/Syne-1 homology (KASH) domain-containing isoforms of nesprin-1 are nuclear envelope-localized transmembrane proteins expressed in select cell types, including RGCs. Of mAKAP's three spectrin repeat domains, the third heterodimerizes with nesprin-1 C-terminal spectrin repeat domains, such that the short isoform nesprin-1α highly expressed in myocytes and adult retina will direct mAKAP to the nuclear envelope when expressed together in cells. We now show that nesprin-1 is present on the nuclear envelope in hippocampal neurons (FIG. 1b), just as we have previously shown for mAKAPα. Overexpression of a GFP-tagged fragment encoding the mAKAP spectrin-repeat domains (amino acid residues 586-1286, “mAKAP-SR-GFP”, FIG. 1a) will displace mAKAPβ from nesprin-1α at the nuclear envelope in myocytes. Expression of mAKAP-SR-GFP similarly displaced DsRed-tagged mAKAPα from the nuclear envelope in neurons (FIG. 1c).

Following our previous publication demonstrating that mAKAPα expression is required for axon growth, we tested whether proper mAKAPα localization was also required for axon growth. Hippocampal neurons were infected with adenovirus expressing the delocalizing mAKAP-SR-GFP peptide in the presence or absence of KCl that induces activity-dependent neurite extension via cAMP and PKA-dependent mechanisms. Outgrowth measurements were performed following 3-5 days in vitro. By this time in culture, both hippocampal (and RGC) neurons are typically polarized with the longest neurite being the elongating axon. Expression of mAKAP-SR-GFP inhibited axon outgrowth as assayed by measurement of longest neurite length, both for neurons cultured in defined media, as well as neurons stimulated by chronic KCl depolarization (FIG. 1d,e). These results suggest that proper localization of the mAKAPα scaffold, and not merely expression (as shown previously in RGCs), is critical for axon extension. These data also support the premise for investigating whether perinuclear cAMP at mAKAPα signalosomes has a unique function in neurons.

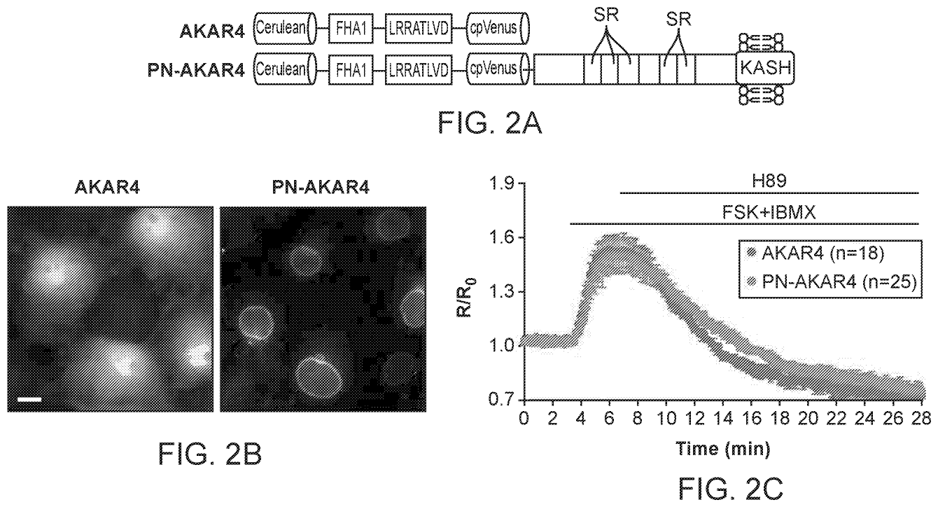

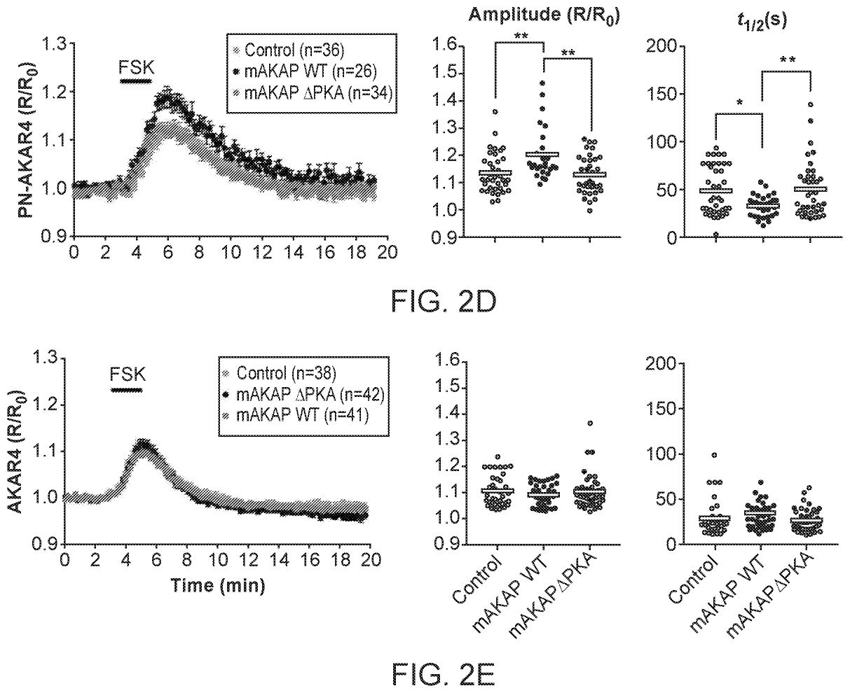

Generation of an mAKAPα-dependent PKA activity sensor. PKA activity in living cells can be assayed with spatiotemporal resolution using genetically encoded FRET biosensors. AKAR4 is a well-characterized biosensor that contains a PKA target site and a FH1 phospho-amino acid-binding domain inserted between donor cerulean and acceptor cpVenus-E172 fluorescent proteins (FIG. 2a), such that sensor phosphorylation increases FRET signal. To assay PKA activity in a mAKAP-specific perinuclear compartment, AKAR4 was expressed in fusion to the N-terminus of nesprin-1α (FIG. 2a). Initial characterization of perinuclear-AKAR4 (PN-AKAR4) was performed in Cos-7 cells, a heterologous cell line that lacks nesprin-1α and mAKAP. When the fusion biosensor was expressed at moderate levels to avoid saturation of the KASH domain-mediated nuclear envelope localization mechanism, cerulean and cpVE172-E172 fluorescence were limited to the nuclear envelope (FIG. 2b). Maximum and minimum FRET signals were obtained for both PN-AKAR4 and AKAR4 by infusing the adenylyl cyclase activator forskolin (FSK) and the phosphodiesterase inhibitor 3-isobutyl-1-methylxanthine (IBMX) followed by the PKA inhibitor H-89, demonstrating that the localized sensor had the same dynamic range of response as the diffusely localized parent sensor (FIG. 2c). Likewise, a 2-minute pulse of FSK resulted in a similar transient increase in PN-AKAR4 FRET signal as AKAR4 sensor (FIG. 2d). Co-expression of mAKAPα resulted in a PN-AKAR4 transient that was ˜50% greater in amplitude and exhibited a more rapid signal decay (t_{1/2}), consistent with the recruitment by mAKAPα of both PKA and PDE4D3 to the nesprin-1α perinuclear compartment. That mAKAPα recruited PKA to nesprin-1α was confirmed by expression of a full-length mAKAPα PKA-binding mutant (mAKAPα ΔPKA) lacking residues 2053-2073 that did not enhance PN-AKAR4 signal. In addition, co-expression of mAKAPα WT (and mAKAPα ΔPKA) did not significantly affect the level of PKA activity detected by the parent sensor (FIG. 2e), consistent with the specific docking of mAKAPα by nesprin-1α.

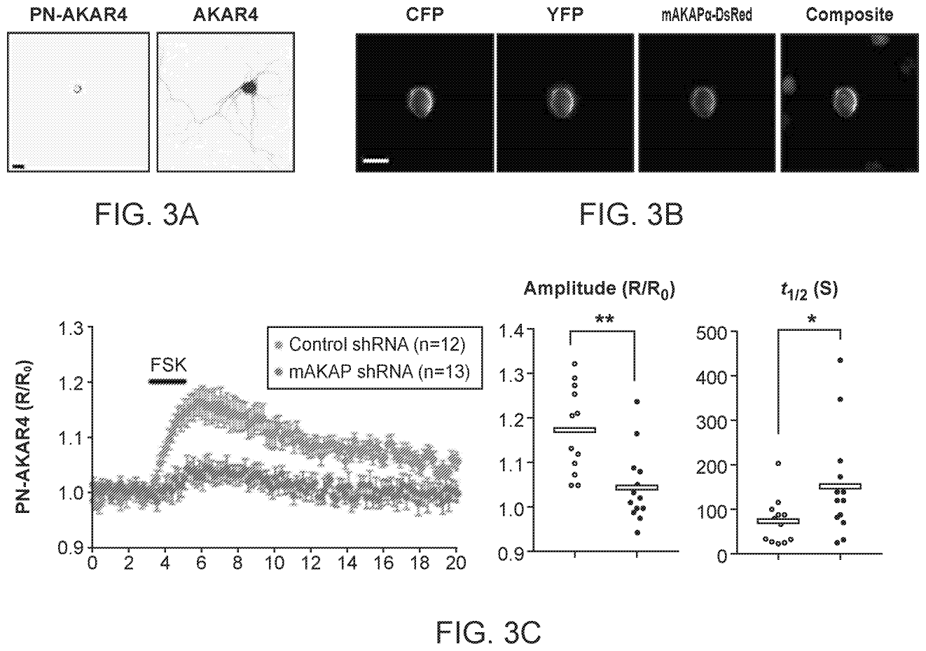

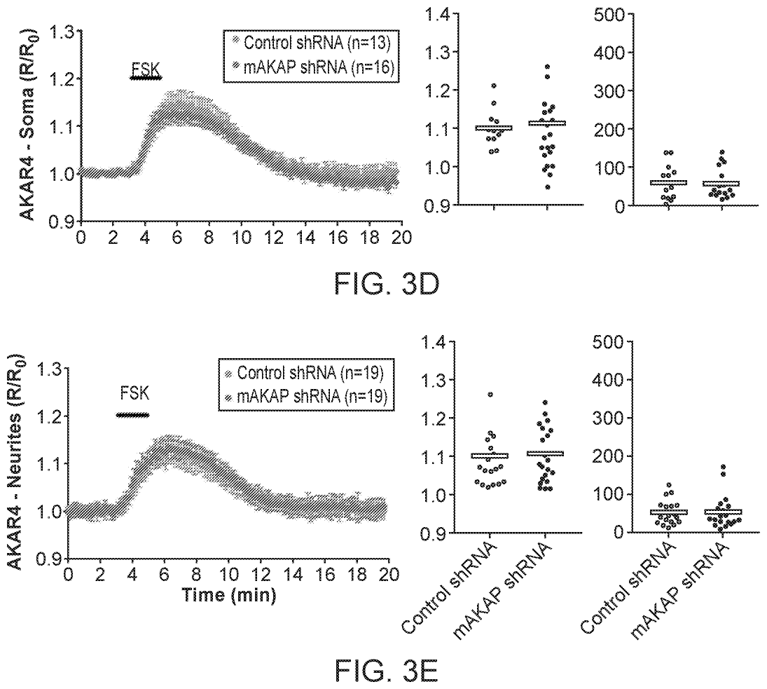

Validation of PN-AKAR4 as a mAKAPα-specific biosensor in hippocampal neurons, where the sensors were similarly localized (FIG. 3a,b), was demonstrated by RNA interference (RNAi) of endogenous mAKAPα expression. FSK stimulation of PN-AKAR4 in neurons resulted in a PKA transient whose amplitude was inhibited ˜75% by co-expression of a mAKAP shRNA and whose signal decay was 2-fold slower (FIG. 3c). Importantly, mAKAPα depletion had no significant effect upon signal detected by the parent AKAR4 present in the soma or the neurites of the neurons (FIG. 3d,e). Taken together with the data obtained in heterologous cells, these results show that PN-AKAR4 is a reporter specific for PKA activity associated with mAKAPα signalosomes at the neuronal nuclear envelope, where mAKAPα signalosome formation affects the kinetics and amplitude of PKA signaling.

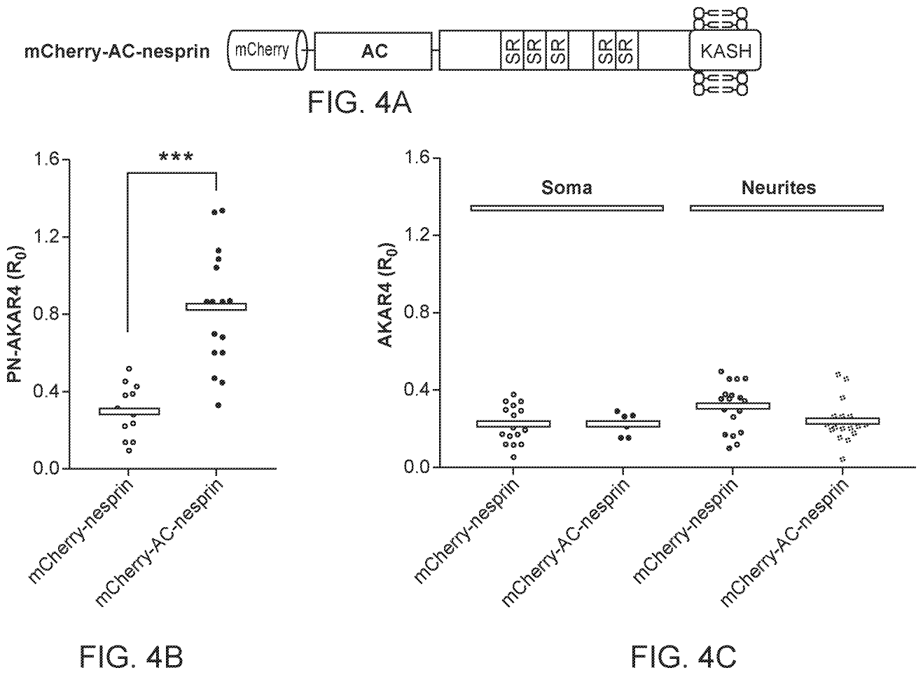

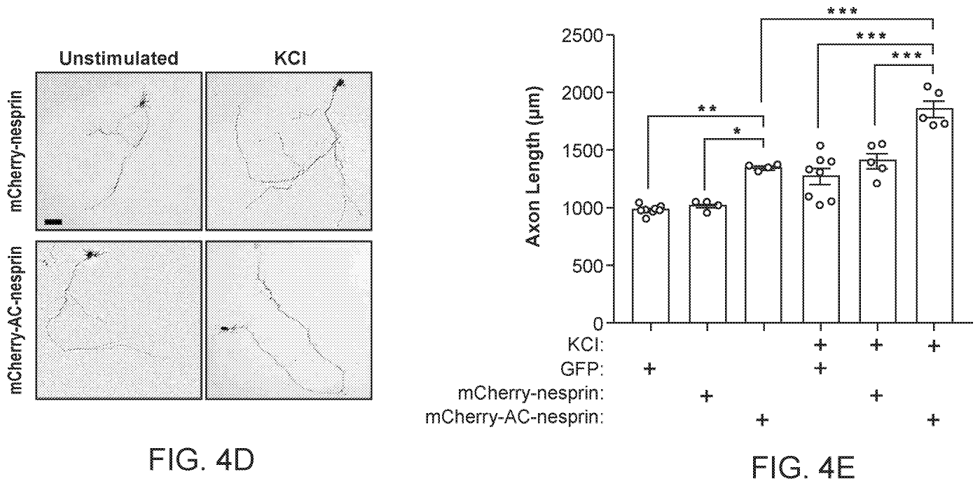

Increased cAMP at mAKAPα promotes neurite extension. FSK stimulation to elevate globally cAMP levels induces axon growth in neurons. To determine whether elevating cAMP specifically at mAKAPα signalosomes is sufficient to induce neurite outgrowth, we constructed an mCherry-tagged protein containing the constitutively-active adenylyl cyclase catalytic domain from soluble adenylyl cyclase (ADCY10) fused to nesprin-1α to locally synthesize cAMP in that compartment (mCherry-AC-nesprin, FIG. 4a). Transient co-expression of mCherry-AC-nesprin with PN-AKAR4 resulted in a 2.7-fold increased baseline FRET signal when compared to mCherry-nesprin control (FIG. 4b). Importantly, expression of mCherry-AC-nesprin had no effect on parent AKAR4 in soma or neurites, showing that the constitutively increased cAMP production was limited to the nesprin/mAKAPα perinuclear compartment (FIG. 4c). Turning to effects on axon growth, measurement of the longest neurite per cell revealed that regardless of co-stimulation by chronic KCl depolarization that promotes axon growth, expression of mCherry-AC-nesprin increased axon extension when compared to neurons expressing control mCherry-nesprin (FIG. 4d, 32% and 31% in the absence and presence of KCl respectively), similarly to that due to KCl depolarization alone (29% and 37% for GFP and mCherry-nesprin-expressing neurons, respectively). These results imply that cAMP signaling spatially restricted to the mAKAPα perinuclear compartment is sufficient to induce hippocampal neuron axon outgrowth.

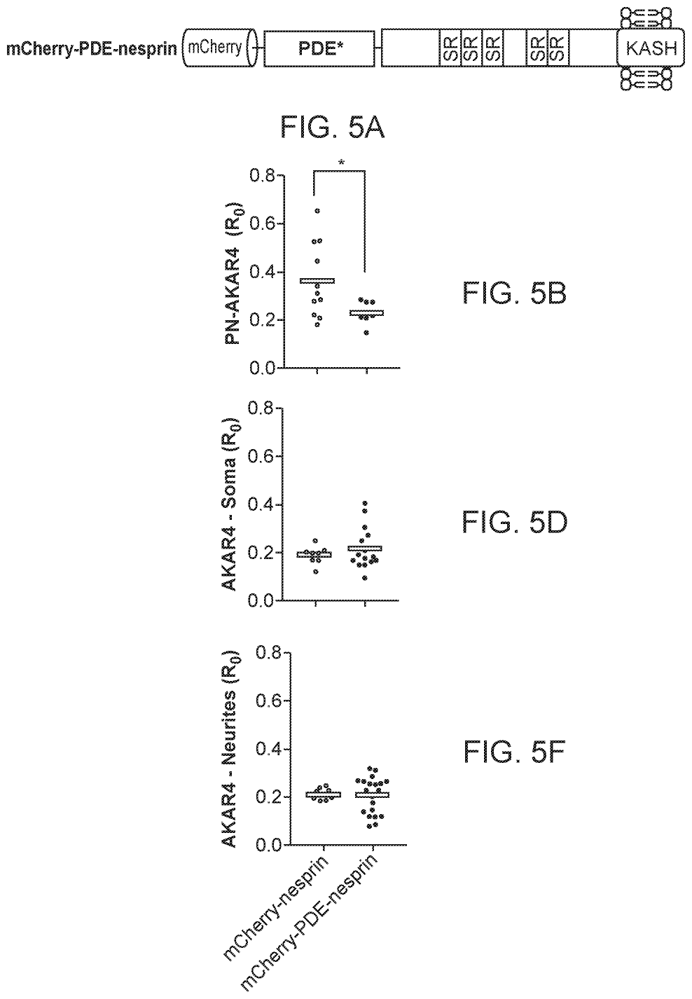

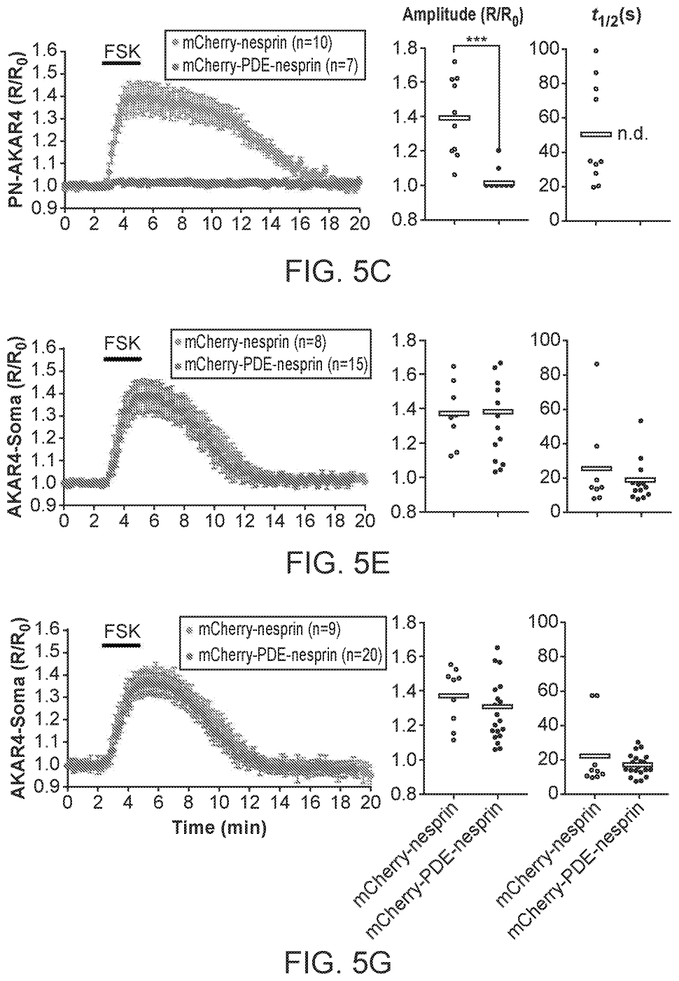

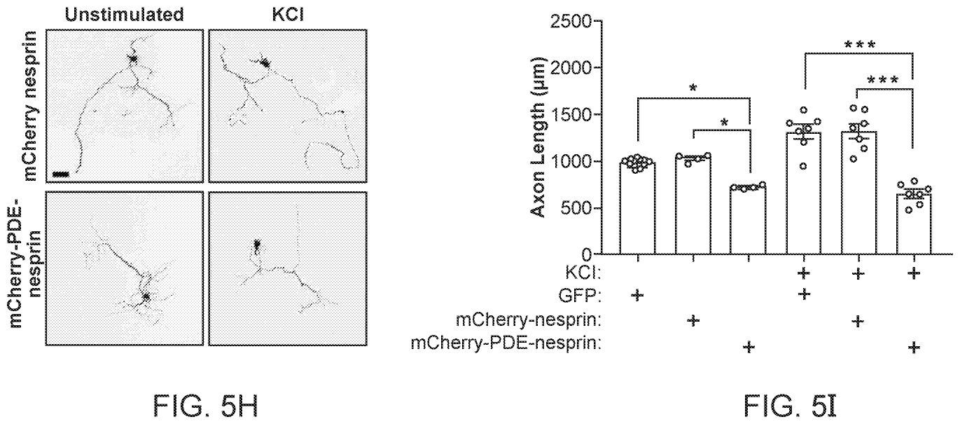

Increased PDE activity at mAKAPα suppresses neurite extension. We next asked the converse question, whether cAMP elevation at the perinuclear compartment is necessary for neurite growth. To prevent cAMP signaling at mAKAPα signalosomes, an mCherry-tagged protein was constructed containing the catalytic domain of PDE4D fused to nesprin-1α (mCherry-PDE-nesprin, FIG. 5a) to constitutively degrade cAMP near the mAKAPα scaffold. ERK mitogen-activated protein kinase (MAP-kinase) can bind and phosphorylate PDE4D, resulting in PDE4D inhibition. To preclude inhibition of the PDE construct by ERK signaling, the KIM and FQF docking sites and Ser-579 phosphorylation site on the PDE4D catalytic domain were ablated by missense mutation. Transient co-expression of mCherry-PDE-nesprin reduced baseline PN-AKAR4 FRET signal 36% in hippocampal neurons when compared to cells expressing control mCherry-nesprin (FIG. 5b). In addition, expression of mCherry-PDE-nesprin completely prevented the induction of a PN-AKAR4 FRET transient by FSK in hippocampal neurons (FIG. 5c). Importantly, increased perinuclear PDE activity had no effect on AKAR4 FRET signals either in the soma or neurites (FIG. 5d-g), demonstrating compartment-specific cAMP depletion.

Consistent with decreased perinuclear cAMP signaling in neurons expressing mCherry-PDE-nesprin, axon growth was significantly inhibited 21% compared to cells expressing control mCherry-nesprin (FIG. 5h). Importantly, chronic KCl depolarization was unable to induce axon extension in cells expressing the mCherry-PDE-nesprin construct indicating that enhanced PDE activity at mAKAPα-nesprin-1α complexes can suppress depolarization-induced growth (p=0.95 for mCherry-PDE-nesprin+/−KCl). Taken together with the above results, these data show that cAMP signaling at perinuclear mAKAPα signalosomes is both sufficient and necessary for neurite extension in cultured hippocampal neurons.

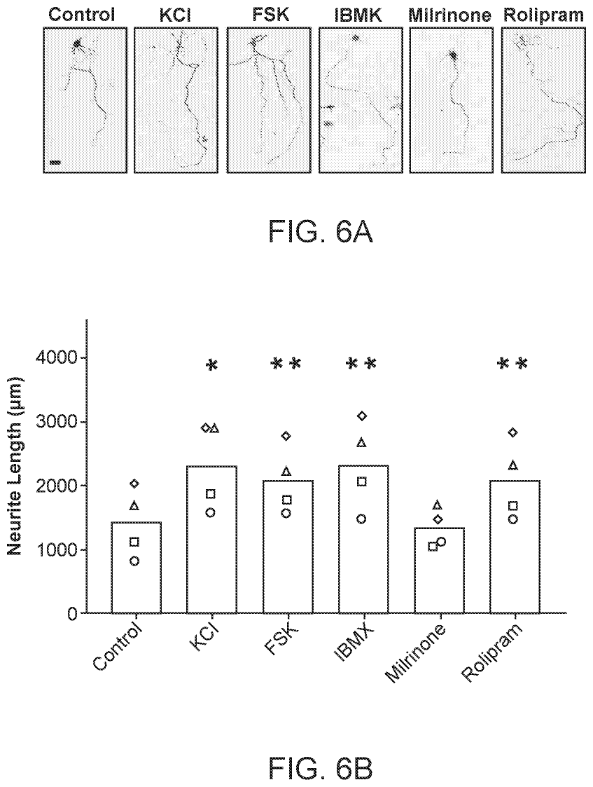

mAKAPα-associated PDE4D3 regulates neurite extension. Given our new findings that mAKAPα-associated perinuclear cAMP regulates neurite extension, we were interested whether inhibition of endogenous mAKAPα-associated PDE activity would similarly promote neurite outgrowth. The mAKAPα scaffold binds a type 4 cAMP-specific phosphodiesterase PDE4D3. First, we tested whether inhibition using the PDE4 inhibitor rolipram would promote neurite extension in hippocampal neurons. Similar to results previously obtained for motoneurons, addition of rolipram to the culture medium induced neurite extension comparably to that with KCl depolarization to induce adenylyl cyclase activity via calcium-dependent signaling, FSK to induce adenylyl cyclase activity directly, and IBMX to inhibit all phosphodiesterases (FIG. 6). Consistent with the recognized differences between PDE3 and PDE4 activity in neurons, PDE3 inhibition with milrinone had no effect on neurite growth.

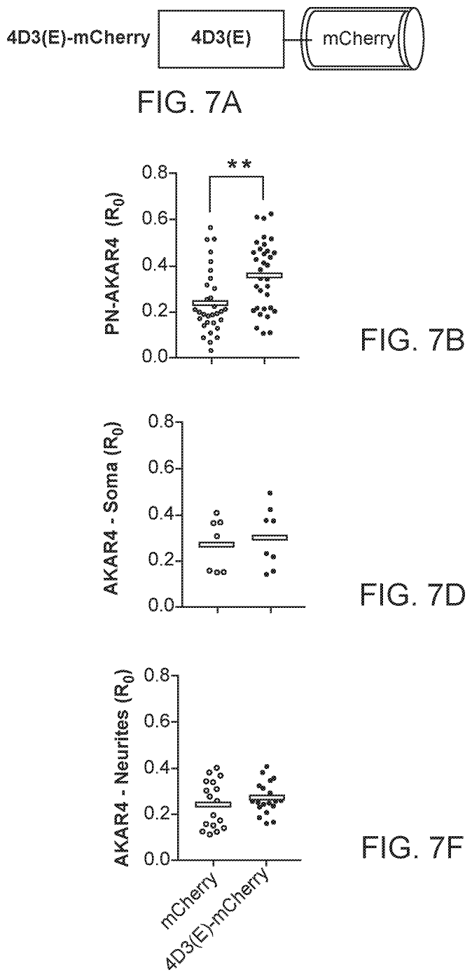

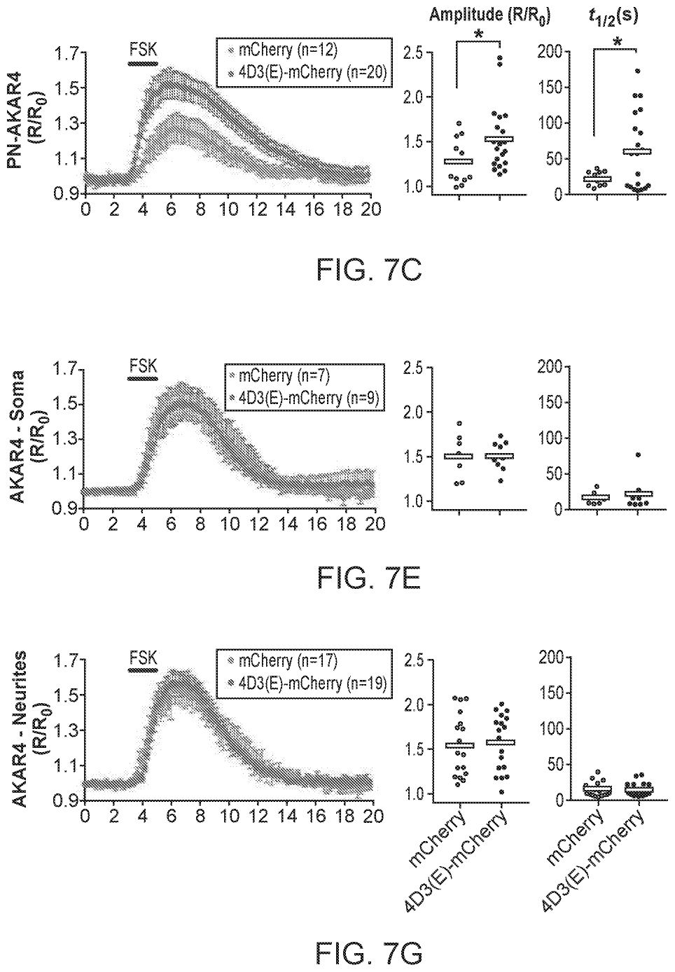

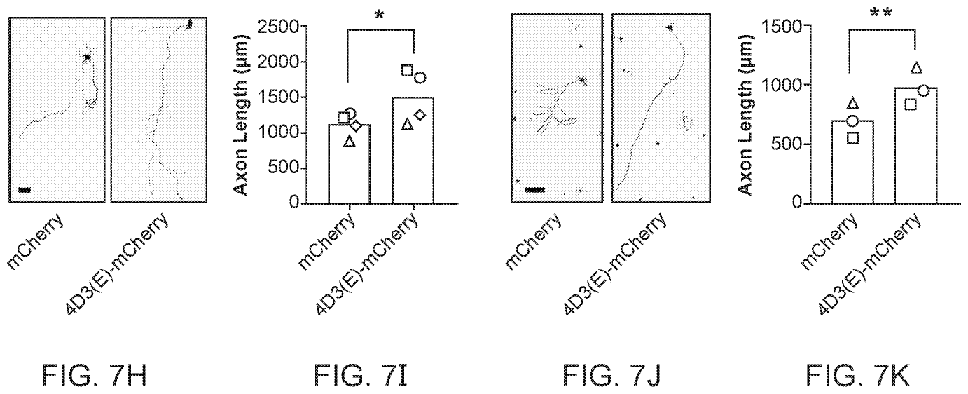

To specifically target and disrupt PDE4D3-mAKAP binding, we took advantage of the fact that the D3-specific N-terminal peptide confers direct binding to mAKAP within residues 1286-1401. We generated a genetically-encoded anchoring disruptor by fusing residues 1-20 of PDE4D3 encoding the D3 peptide via a flexible linker (ELAAK×3) to the N-terminus of mCherry (FIG. 7a). PDE4D3 binding to mAKAP is enhanced by PKA phosphorylation of residue Ser-13 that can be mimicked by Ser to Glu substitution. To increase the affinity and potency of the anchoring disruptor, the mCherry fusion peptide included this Ser¹³Glu missense mutation, “4D3(E)”. Consistent with our hypothesis that displacement of PDE4D3 from mAKAPα signalosomes should increase cAMP persistence and PKA activity, expression of 4D3(E)-mCherry increased baseline FRET signal 1.5-fold when compared to mCherry control (FIG. 7b), and potentiated the PN-AKAR4 FRET response to FSK pulse in hippocampal neurons (2-fold, FIG. 7c). Importantly, even though the 4D3(E)-mCherry peptide was diffusely expressed throughout the cell (cf. panels h and j), it had no significant effect on FRET signals obtained with the parent AKAR4 in the hippocampal neuron soma or neurites (FIG. 7d-g). Furthermore, similar to the effects of mCherry-AC-nesprin increasing cAMP/PKA at the perinuclear compartment, 4D3(E)-mCherry peptide expression increased axon extension ˜35% in hippocampal neurons (FIG. 7h,i). Notably, 4D3(E)-mCherry peptide expression increased axon extension ˜40% in RGCs as well (FIG. 7j,k). These results indicate that PDE4D3 at perinuclear mAKAPα in neurons regulates cAMP signals and PKA activity in that compartment and demonstrates conservation of PDE4D3 function among two mAKAPα-expressing central nervous system neuronal cell types.

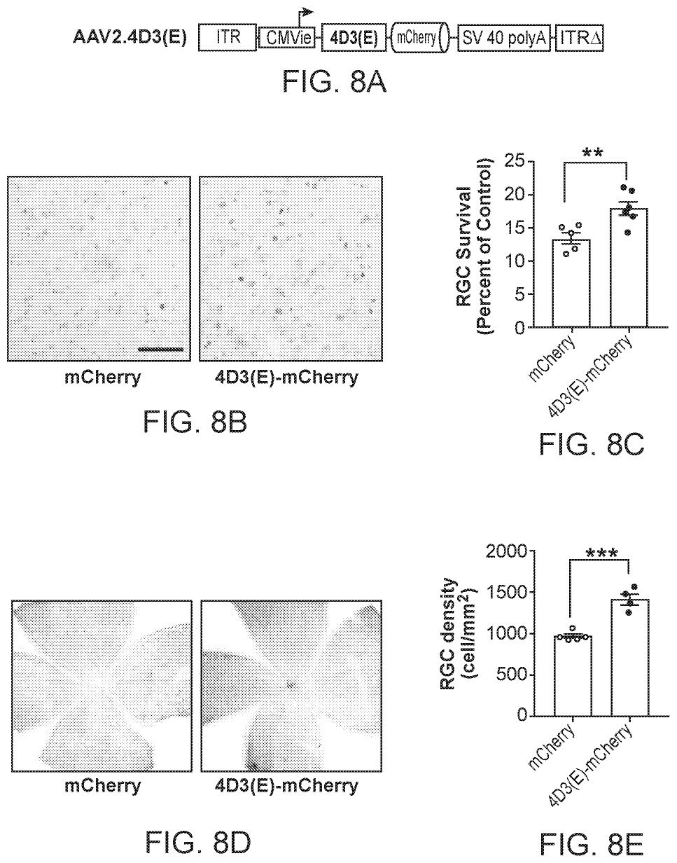

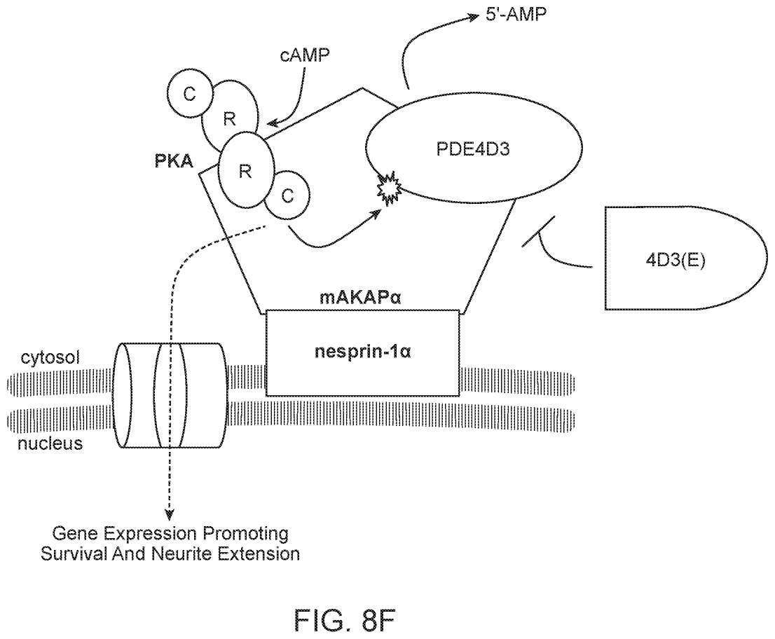

RGC survival after optic nerve crush is enhanced by PDE4D3 anchoring disruption. Previously we showed that in addition to axon growth in vitro, expression of the mAKAPα scaffold is required for the beneficial effects of exogenous neurotrophic factor and cAMP-analogs for the survival of RGC neurons following optic nerve crush injury. Intravitreal AAV2 preferentially transduces RGCs, allowing cell-type selective gene delivery and peptide expression. To test in vivo whether cAMP specifically localized at mAKAPα signalosomes functions as an autonomous signaling compartment promoting neuroprotection or axon regeneration, we injected mice intravitreally with AAV2 for 4D3(E)-mCherry peptide or mCherry control (FIG. 8a). Anterograde labeling of RGC axons 24 hours before euthanasia revealed that PDE4D3 displacement did not promote axonal regeneration within 2 weeks after optic nerve crush. However, staining of retina for the RGC-specific marker RNA binding protein with multiple splicing (RBPMS) revealed that RGC survival was increased 30-50% with the 4D3 displacing peptide compared to control AAV2.mCherry alone, whether in comparison to the control uncrushed contralateral eye (FIG. 8b,c) or in an independent experiment with a different masked investigator and quantified as absolute RGC cell density (FIG. 8d,e). These results show that PDE4D3-anchoring disruption provides a method for enhancing physiologically relevant cAMP signaling in the mAKAPα signaling compartment that promotes neuronal survival after injury.

### Discussion

Using a series of new molecular tools to induce or suppress cAMP levels at the mAKAPα scaffold, we have demonstrated that cAMP signaling at mAKAPα perinuclear signalosomes constitutes a unique signaling compartment within neurons that regulates both neuronal survival in vivo and axon growth in vitro. Despite obvious PKA activity in the soma, suppression of cAMP levels exclusively at mAKAPα signalosomes via expression of mCherry-PDE-nesprin prevented baseline and depolarization-induced axon growth. Conversely, elevating cAMP levels at mAKAPα using mCherry-AC-nesprin was sufficient to induce axon growth. The use of PKA activity reporters in these experiments not only validated the new molecular tools as specific for perinuclear signaling, but also provide evidence for cAMP and PKA compartmentation. Live cell imaging using nesprin-1α-localized and parent AKAR4 FRET biosensors showed that even though mAKAPα signalosomes are localized to the nuclear envelope where there should be no physical barrier to cAMP diffusion, local perinuclear production of cAMP sufficient to alter cellular phenotype did not result in a detectable increase in PKA activity elsewhere in the soma or neurites. These results support the general model in which PKA action is spatially restricted by AKAP signalosomes that may autonomously control local cAMP fluxes. This spatial specificity is critical given the large number of cellular processes regulated by cAMP and PKA, including various steps in the formation of neural connectivity in which cAMP signaling can sometimes play opposing roles.

How is cAMP spatially restricted in the perinuclear compartment? Type 4 PDE is a major source of cAMP degrading activity in neurons and is likely important for establishing cAMP compartmentation. PDE4 isoforms are distinguished by their individual N-terminal peptides that target them to different intracellular locations, and mAKAPα binds only type 4D3 PDE through the N-terminal D3 peptide. Displacement of individual signaling enzymes from signalosomes using anchoring disruptor peptides is an approach that allows both the testing of specific enzyme function and the selective modification of signalosome function without affecting global cellular signaling as often occurs with enzyme catalytic inhibitors. Consistent with a role for mAKAPα-dependent cAMP signaling in axon growth, enhancement of PN-AKAR4 signal by PDE4D3 displacement using 4D3(E)-mCherry correlated with increased axon growth in vitro. Overall, these results indicate that PDE4D3 associates with mAKAP signalosomes and limits the associated cAMP signals at that nuclear envelope compartment.

We have previously shown that RGC-specific knock-out of mAKAPα expression blocked the neuroprotective effects of CPT-cAMP and brain-derived neurotrophic factor (BDNF) after optic nerve crush. While it is unclear whether the roles of mAKAPα in axon extension and neuroprotection involve the same downstream effectors, enhanced cAMP signaling at mAKAPα by PDE4D3 displacement also increased RGC survival after optic nerve injury in vivo. In fact, we found that AAV-mediated expression of 4D3(E)-mCherry was as effective at preserving RGC numbers as previously reported for exogenous CPT-cAMP intravitreal injection, although we did not directly compare these in the current studies. It should also be noted that in these experiments 4D3(E)-mCherry did not promote axon regeneration, consistent with previous findings that cAMP signaling alone does not significantly promote axon regeneration in the absence of additional intervention. Taken together, our data show that specific anchoring disruption of a relevant single PDE isoform is sufficient to promote PKA signaling and alter cellular phenotype in a manner consistent with the function of the corresponding scaffold protein.

cAMP-dependent signaling is relevant to formation of the neuronal cell networks during development as well as survival and regeneration in the adult after injury. The formation of neuronal connections involves multiple cAMP-dependent steps, including polarization of immature neurons, axon elongation and branching, axon target guidance, and pruning of inappropriate synapses. cAMP and PKA activity gradients have been found in hippocampal neurons, with significantly higher levels in the distal axon of mature neurons. In addition, a cAMP compartment at plasma membrane lipid rafts has been shown to be important for ephrin-A regulated axonal pruning. mAKAPα-associated cAMP signaling is unlikely to be relevant to all of the different steps in neuronal development, but due to its perinuclear location is poised to regulate gene expression through the post-translational modification of transcription factors and histone deacetylases that might regulate specific aspects of the overall program (FIG. 8c). It is well-established that signaling by cAMP, including that produced by soluble adenylyl cyclase and mediated by PKA, is required for activity-dependent axon growth. Our results show that cAMP signaling at perinuclear mAKAPα signalosomes promotes neurite outgrowth independently of KCl stimulation, whether as part of a regulatory pathway in parallel or in series with that induced by depolarization.

mAKAPα signalosomes may selectively regulate gene expression that enables increased axonal growth and promotes neuroprotection after injury, the localization, kinetics and effects of which are defined by PDE4D3 and PKA. It has been recently reported that activity-induced elevation of cAMP in injured RGCs potentiates the effects of growth promoting manipulations including mTOR activation.

The significance of these results extends beyond a demonstration of mAKAPα signalosome compartmentation and function. Loss of RGCs is a critical factor contributing to vision loss in many eye diseases, including in glaucoma which is expected to affect ˜80 million people worldwide by 2020, of whom ˜10% are predicted will go blind. Given the great promise for AAV-based human ophthalmic therapies, AAV-based 4D3(E) anchoring disruptor expression provides a treatment of RGC neurodegenerative diseases.

### Methods

Plasmid constructs. A description of relevant plasmids and viruses is provided below. Additional details and complete vector maps for all constructions are available upon request. Many of these plasmids were constructed by Genewiz using methods of the company's choice. Plasmid constructs were validated by sequencing and by expression of the encoded recombinant proteins in Cos-7 cells. The “pS” series of vectors in which the conditional tetracycline-responsive promoter has been replaced with the CMV immediate early promoter are adenoviral shuttle vectors based upon the pTRE vector (Clontech) containing I-Ceu I and PI-Sce I sites for subcloning into the adenovirus bacterial vector Adeno-X (Clontech). Adenovirus was purified after amplification using Vivapure AdenoPACK kits (Sartorium) and titered using HEK293 cells. AAV were produced by the University of Pennsylvania Vector Core with funding provided in part by the NHLBI Gene Therapy Resource Program.

pS-mCherry-PDE4D_C(ERK−)-nesprin expression plasmid includes a cDNA expressing the following protein fragments: mCherry—human PDE4D3 catalytic domain (aa 225-673, NP_006194.2) with missense mutations K455A/K456A/S579A/F597A/Q598A/F599A—myc tag—human nesprin-1α (AAN60442.1 aa 7799-8797). pS-mCherry-AC-nesprin contains a rat soluble adenylyl cyclase C1+C2 domains fragment (NP067716.1 aa 1-469) replacing the PDE BsrGI-Not I fragment of pS-mCherry-PDE4D_C(ERK−)-nesprin. pS-mCherry-nesprin control vector is the same as the above vectors except lacking an EcoRI-Xho I fragment containing the AC or PDE domain and myc-tag sequences.

pscS2-4D3(E)-mCherry-mh is a shuttle vector for both subcloning into adenovirus and for directly producing self-complementary AAV, containing the following: 1) AAV2 (NC_001401.2) bp 4664-4489 in antisense orientation 5′ to a PI-Sce I sites; 2) CMV immediate early promoter; 3) a cDNA expressing human PDE4D3 (1-20) with S13E mutation-(ELAAK)₃ flexible linker-mCherry-myc tag-His₆ tag fusion protein; 4) SV40 poly A sequence; and 5) AAV2 bp (NC_001401.2) 4559-4662 3′ to a I-Ceu I site. pscS2-mCherry-mh control vector was constructed by deleting a Nhe I-Age I fragment of pscS2-4D3(E)-mCherry-mh that encodes the 4D3(E) peptide.

The FRET based PKA sensor AKAR4 in pcDNA3 was kindly provided by Dr. Jin Zhang (Johns Hopkins University). pS-AKAR4 adenoviral shuttle vector was constructed by subcloning the AKAR4 cDNA from pCDNA3-AKAR4 into the NheI and PspOMI sites of pS-mCherry-Nesprin. The shuttle vector pS-AKAR4-Nesprin1α encodes PN-AKAR4 that includes human nesprin-1α (AAN60442.1 aa 7799-8797) at the C-terminus of AKAR4.

Plasmids and adenovirus for rat mAKAP and control shRNA and encoding myc-tagged mAKAP 586-1286 (myc-mAKAP-SR) were as previously described. Adenovirus expressing N-terminally myc-tagged rat mAKAPα were generated using a pTRE (Clontech) expression vector containing a cDNA with a myc-tag followed by a full-length mAKAPα open reading frame (NM_022618.1 bp 128-7138). mAKAPα PKA was expressed using adenovirus containing a deletion of mAKAP base pairs 6284-6346 (codons 2053-2073). Expression vector GFP-PDE4D3-vsv was a previously described.

Antibodies

| Antigen | Species and Catalog number | Company |
|---|---|---|
| βIII-tubulin | | |
| GFP | Rabbit sc-8334 | Santa Cruz |
| GFP | Chicken ab13972 | Abcam |
| Flag tag | Rabbit F7425 | Sigmal-Aldrich |
| HA tag | Mouse HA-7 monoclonal | Sigma-Aldrich |
| Myc tag | Mouse 4A6 monoclonal | Millipore |
| Myc tag | Rabbit 06-549 | Millipore |
| nesprin-1 | Mouse MANNES1A(7A12) | Invitrogen |
| mAKAP | VO54 | Kapiloff lab |
| MAP2 | Mouse MAB3418 | Millipore |
| RBPMS | Guinea Pig | Gift from Hu |
| | | Lab |

Animal Models. All in vivo research was performed under the supervision of the Institutional Animal Care and Use Committee at the University of Miami or Stanford University. All rats used in this project were Sprague-Dawley, and all mice used in this project were C57BL/6.

### Cell Culture

Cos-7 cells: Cos-7 cells were maintained in DMEM (10% v/v FBS) at 37° C. in a humidified incubator with 5% CO₂. For live cell imaging, Cos-7 cells were plated onto 25-mm diameter sterilized glass coverslips in 6-well plates and were either transfected with JPEI or infected with adenovirus at 60-70% confluence and allowed to grow for 24-48 h before live cell imaging. Nesprin-1α fusion proteins are not properly localized to the nuclear envelope when grossly over-expressed due to saturation of KASH-SUN domain protein-protein interactions. Only Cos-7 cells and neurons with epifluorescence for PN-AKAR and the other nesprin-1α fusion proteins restricted to the nuclear envelope were included in the studies.

Primary rat hippocampal neurons: Hippocampal cultures were prepared from Sprague Dawley rat embryonic day 18 embryos. Briefly, the rat hippocampal CA1-CA3 region was dissected in PBS medium with 10 mM D-glucose and digested with 0.05% trypsin-EDTA in PBS with 11 mM D-glucose for 30 min at 37° C. The dissociated tissues were centrifuged at 250 g for 2 min and then triturated with fire polished glass pipet in Hank's balanced salt solution (HBSS) with calcium and magnesium in plating medium (10% v/v horse serum in DMEM). Dissociated neurons were plated on nitric acid-treated 25-mm cover glass coated with poly-L-lysine in plating medium. Four hours after plating, the medium was replaced with maintenance medium supplemented with 1% N2, 2% B27 (Invitrogen, Carlsbad, Calif., USA), 5 mM D-glucose, 1 mM sodium pyruvate. Four days later, 4 μM arabinosyl cytosine was added to inhibit glial proliferation and the neurons were either transfected with JPEI or infected with adenovirus.

Live cell imaging was performed 36-72 h after transfection/infection as described below. For neurite extension assays, the cells were cultured for two days in DMEM with 1 μg/ml chicken egg albumin and 1 mM sodium pyruvate. 40 mM KCl, 10 μM FSK, 100 μM IBMX, 20 μM Milrinone, 10 μM Rolipram were included as indicated. Two days later, the neurons were fixed and stained with antibodies. Nuclei were counter stained with DAPI and SlowFade Gold antifade solution (Molecular Probes) was added before coverslip mounting. Images were acquired with IPLab or Slidebook 6 by wide-field microscopy (Leica DMI 6000B or Zeiss Axio Observer 7) and processed with Adobe PhotoShop CSS 12.1. The length of the longest neurite for ˜15 neurons average per condition was measured for each experiment with ImageJ with Simple Neurite Tracer plugin. Other neuronal images were acquired using a Zeiss 880 confocal microscope.

Retinal Ganglion Cells: RGCs were purified (N99.5%) from postnatal (P2 to P4) Sprague-Dawley rats through sequential immunopanning, as previously described. Following purification, RGCs were seeded at 1000-2500 cells/well in poly-D-lysine (PDL; 70 kDa, 10 μg/mL; Sigma, St. Louis, Mo.) and laminin (1 μg/mL; Invitrogen, Carlsbad, Calif.) coated 24 well plates. RGCs were cultured in neurobasal (NB) serum-free defined medium containing insulin (5 μg/mL), sodium pyruvate (1 mM), L-glutamine (1 mM), triiodothyronine (T3; 40 ng/mL; Sigma), N-acetyl cysteine (NAC; 5 μg/mL; Sigma), B27 (1:50), BDNF (50 ng/ml), CNTF (10 ng/ml) and FSK (5 μM) as described. 4 hours after seeding, RGCs were incubated with AAV2-4D3-mCherry or -mCherry viral particles at 75,000 MOI for 1 hour followed by a half media change and a full media change the next day. After an additional three days incubation in 10% CO₂ at 37° C., the RGCs were fixed and stained for βIII-tubulin. Images were acquired on a Zeiss Axio Observer inverted microscope and the longest neurite per cell (˜15 cells average per condition per experiment) measured using ImageJ Neurite Tracer. Dead, overlapping and mCherry-negative RGCs were excluded from the analysis.

Live cell FRET imaging. Live cell images were acquired using either 1) a DMI6000B inverted microscope (Leica) with 63× Plan Apo/1.25 HCX PL FLUOTAR objective, and LB10-NWIQ component (fluorescent light source, filter wheel, ultrafast shutter, Leica) and Qimaging Retiga EXi camera driven by Slidebook 6.0. or 2) automated, inverted Zeiss Axio Observer 7 Marianas™ Microscope equipped with a X-Cite 120LED Boost White Light LED System and a high-resolution Prime™ Scientific CMOS digital camera that is controlled by a workstation loaded with SlideBook imaging and microscope control software (Intelligent Imaging Innovations, Inc.). Filters were as follows: Dichroic—FF459/526/596-Di01; CFP Exciter—FF02-438/24; CFP Emitter—FF01-482/25; YFP Exciter—FF01-509/22; YFP Emitter—FF01-544/24; mCherry Exciter—FF01-578/21; mCherry Emitter—FF02-641/75. Cells were washed twice before imaging in PBS with 11 mM D-glucose and perfused during imaging with Tyrode solution (137 mM NaCl, 2.7 mM KCl, 1 mM MgCl₂, 2 mM CaCl₂), 0.2 mM Na₂HPO₄, 12 mM NaHCO₃, 11 mM D-glucose, 25 mM HEPES, 1% BSA) at room temperature (23-25° C.) in a perfusion chamber (Warner Instruments). For stimulation of cells, the bath solution was exchanged by peristaltic pump (Harvard Apparatus) perfusion with different drugs in Tyrode solution. During live cell imaging the exposure time for FRET, acceptor and donor channels was 100 ms and images were collected every 10 s. Baseline images were acquired for 2-5 min. All imaging processing was performed using Slidebooks software. Net FRET for regions of interest was calculated by subtracting bleedthrough for both the donor and acceptor channels. FRET ratio “R” was net FRET÷ background subtracted donor signal, with R₀ the ratio for time=0.

Intravitreal Injection and Optic Nerve Crush. AAV2-4D3-mCherry or -mCherry control (2 μL 5-7×10¹² vg/ml) was injected intravitreally into adult P20-P30 wildtype mice 2 weeks prior to optic nerve crush. Intravitreal injections were performed just posterior to the pars plana with a 31-gauge needle (Hamilton) connected to a 5 μL Hamilton syringe. Care was taken not to damage the lens. For nerve crush, the left optic nerve (ON) was exposed from the outer canthus and crushed for 5 s with a Dumont #5 forceps (91150-20, F.S.T.) approximately 1.5 mm behind the globe. Care was taken to avoid damaging the blood supply to the retina. Mice with any significant postoperative complications (e.g., retinal ischemia, cataract) were excluded from further analysis.

Two weeks after optic nerve crush, mice were euthanized by intracardial perfusion with 4% PFA. Retinal flatmount was prepared as described previously. Briefly, the eyes were removed and post-fixed with 4% PFA for 2 h at room temperature. Retinas were flat mounted in mounting medium (ProLong Gold Anti-Fade) on glass slides and stained with RBPMS antibodies. Confocal images were acquired with a confocal laser scanning microscope (Zeiss 880; Zeiss) and a ×10 magnification lens. The imaging and quantification were performed in a masked fashion as previously described. Briefly, the retinas were divided into 4 quadrants, and one digital micrograph was taken from a fixed distance from the periphery of each of the 4 fields. Although mCherry epifluorescence was not evenly distributed throughout the retina, RBPMS-positive RGCs were counted regardless of the apparent level of AAV-based expression.

Statistics. Statistics were computed using Graphpad Prism 7. All unpaired data are expressed as mean±s.e.m. Error bars are not provided for bar graphs showing paired experiments or scatter plots. Repeated symbols are used as follows: single—p≤0.05; double—p≤0.01; triple—p≤0.001. Single comparisons were by two-tailed student t-tests, paired or unpaired as appropriate. All datasets involving multiple comparisons for which p-values are provided were significant by ANOVA, α=0.05. One-way or two-way ANOVA was performed with matching as appropriate. p-values for experiments involving multiple comparisons were obtained by Tukey post-hoc testing, albeit p-values for not all comparisons are indicated on the graphs.

### REFERENCES

1. Langeberg, L. K. & Scott, J. D. Signalling scaffolds and local organization of cellular behaviour. Nat Rev Mol Cell Biol 16, 232-244, (2015).
2. Terenzio, M., Schiavo, G. & Fainzilber, M. Compartmentalized Signaling in Neurons: From Cell Biology to Neuroscience. Neuron 96, 667-679, (2017).
3. Wild, A. R. & Dell'Acqua, M. L. Potential for therapeutic targeting of AKAP signaling complexes in nervous system disorders. Pharmacol. Ther. 185, 99-121, (2018).
4. Averaimo, S. & Nicol, X. Intermingled cAMP, cGMP and calcium spatiotemporal dynamics in developing neuronal circuits. Front Cell Neurosci 8, 376, (2014).
5. Stiles, T. L., Kapiloff, M. S. & Goldberg, J. L. The role of soluble adenylyl cyclase in neurite outgrowth. Biochim. Biophys. Acta 1842, 2561-2568, (2014).
7. Pare, G. C., Easlick, J. L., Mislow, J. M., McNally, E. M. & Kapiloff, M. S. Nesprin-1alpha contributes to the targeting of mAKAP to the cardiac myocyte nuclear envelope. Exp. Cell Res. 303, 388-399, (2005).
8. Michel, J. J. et al. Spatial restriction of PDK1 activation cascades by anchoring to mAKAPalpha. Mol. Cell 20, 661-672, (2005).
9. Lee, S. W. et al. AKAP6 inhibition impairs myoblast differentiation and muscle regeneration: Positive loop between AKAP6 and myogenin. Sci Rep 5, 16523, (2015).
10. Kapiloff, M. S., Schillace, R. V., Westphal, A. M. & Scott, J. D. mAKAP: an A-kinase anchoring protein targeted to the nuclear membrane of differentiated myocytes. J. Cell Sci. 112 Pt 16), 2725-2736, (1999).
11. Kapiloff, M. S. et al. An adenylyl cyclase-mAKAPbeta signaling complex regulates cAMP levels in cardiac myocytes. J. Biol. Chem. 284, 23540-23546, (2009).
12. Dodge, K. L. et al. mAKAP assembles a protein kinase A/PDE4 phosphodiesterase cAMP signaling module. EMBO J. 20, 1921-1930, (2001).
13. Passariello, C. L., Li, J., Dodge-Kafka, K. & Kapiloff, M. S. mAKAP—a master scaffold for cardiac remodeling. J. Cardiovasc. Pharmacol. 65, 218-225, (2015).
14. Dodge-Kafka, K. L., Gildart, M., Li, J., Thakur, H. & Kapiloff, M. S. Bidirectional regulation of HDAC5 by mAKAPbeta signalosomes in cardiac myocytes. J. Mol. Cell. Cardiol. 118, 13-25, (2018).
15. Kritzer, M. D. et al. The scaffold protein muscle A-kinase anchoring protein beta orchestrates cardiac myocyte hypertrophic signaling required for the development of heart failure. Circulation. Heart failure 7, 663-672, (2014).
16. Li, J., Vargas, M. A., Kapiloff, M. S. & Dodge-Kafka, K. L. Regulation of MEF2 transcriptional activity by calcineurin/mAKAP complexes. Exp. Cell Res. 319, 447-454, (2013).
17. Berkelaar, M., Clarke, D. B., Wang, Y. C., Bray, G. M. & Aguayo, A. J. Axotomy results in delayed death and apoptosis of retinal ganglion cells in adult rats. J. Neurosci. 14, 4368-4374, (1994).
18. Zhou, C., Rao, L., Shanahan, C. M. & Zhang, Q. Nesprin-1/2: roles in nuclear envelope organisation, myogenesis and muscle disease. Biochem. Soc. Trans. 46, 311-320, (2018).
19. Razafsky, D. S., Ward, C. L., Kolb, T. & Hodzic, D. Developmental regulation of linkers of the nucleoskeleton to the cytoskeleton during mouse postnatal retinogenesis. Nucleus 4, 399-409, (2013).
20. Goldberg, J. L. et al. Retinal ganglion cells do not extend axons by default: promotion by neurotrophic signaling and electrical activity. Neuron 33, 689-702, (2002).
21. Gorshkov, K. et al. AKAP-mediated feedback control of cAMP gradients in developing hippocampal neurons. Nat Chem Biol 13, 425-431, (2017).
22. Zhou, X., Herbst-Robinson, K. J. & Zhang, J. Visualizing dynamic activities of signaling enzymes using genetically encodable FRET-based biosensors from designs to applications. Methods Enzymol. 504, 317-340, (2012).
23. Depry, C., Allen, M. D. & Zhang, J. Visualization of PKA activity in plasma membrane microdomains. Mol Biosyst 7, 52-58, (2011).
24. Pare, G. C. et al. The mAKAP complex participates in the induction of cardiac myocyte hypertrophy by adrenergic receptor signaling. J. Cell Sci. 118, 5637-5646, (2005).
25. Klussmann, E. Protein-protein interactions of PDE4 family members—Functions, interactions and therapeutic value. Cell. Signal. 28, 713-718, (2016).
26. MacKenzie, S. J., Baillie, G. S., McPhee, I., Bolger, G. B. & Houslay, M. D. ERK2 mitogen-activated protein kinase binding, phosphorylation, and regulation of the PDE4D cAMP-specific phosphodiesterases. The involvement of COOH-terminal docking sites and NH2-terminal UCR regions. J. Biol. Chem. 275, 16609-16617, (2000).
27. Aglah, C., Gordon, T. & Posse de Chaves, E. I. cAMP promotes neurite outgrowth and extension through protein kinase A but independently of Erk activation in cultured rat motoneurons. Neuropharmacology 55, 8-17, (2008).
28. Carlisle Michel, J. J. et al. PKA-phosphorylation of PDE4D3 facilitates recruitment of the mAKAP signalling complex. Biochem. J. 381, 587-592, (2004).
29. Park, K. K. et al. Promoting axon regeneration in the adult CNS by modulation of the PTEN/mTOR pathway. Science 322, 963-966, (2008).
30. Rodriguez, A. R., de Sevilla Muller, L. P. & Brecha, N. C. The RNA binding protein RBPMS is a selective marker of ganglion cells in the mammalian retina. J. Comp. Neurol. 522, 1411-1443, (2014).
31. Kurimoto, T. et al. Long-distance axon regeneration in the mature optic nerve: contributions of oncomodulin, cAMP, and pten gene deletion. J. Neurosci. 30, 15654-15663, (2010).
32. Averaimo, S. et al. A plasma membrane microdomain compartmentalizes ephrin-generated cAMP signals to prune developing retinal axon arbors. Nature communications 7, 12896, (2016).
33. Lim, J. H. et al. Neural activity promotes long-distance, target-specific regeneration of adult retinal axons. Nat Neurosci 19, 1073-1084, (2016).
34. Quigley, H. A. & Broman, A. T. The number of people with glaucoma worldwide in 2010 and 2020. Br. J. Ophthalmol. 90, 262-267, (2006).
35. Auricchio, A., Smith, A. J. & Ali, R. R. The Future Looks Brighter After 25 Years of Retinal Gene Therapy. Hum. Gene Ther. 28, 982-987, (2017).
36. Dodge-Kafka, K. L. et al. cAMP-stimulated protein phosphatase 2A activity associated with muscle A kinase-anchoring protein (mAKAP) signaling complexes inhibits the phosphorylation and activity of the cAMP-specific phosphodiesterase PDE4D3. J. Biol. Chem. 285, 11078-11086, (2010).
37. Meyer-Franke, A., Kaplan, M. R., Pfrieger, F. W. & Barres, B. A. Characterization of the signaling interactions that promote the survival and growth of developing retinal ganglion cells in culture. Neuron 15, 805-819, (1995).
38. Zhao, Z. et al. gammadelta T cells as a major source of IL-17 production during age-dependent RPE degeneration. Invest. Ophthalmol. Vis. Sci. 55, 6580-6589, (2014).
39. Vargas, M. A., Tirnauer, J. S., Glidden, N., Kapiloff, M. S. & Dodge-Kafka, K. L. Myocyte enhancer factor 2 (MEF2) tethering to muscle selective A-kinase anchoring protein (mAKAP) is necessary for myogenic differentiation. Cell. Signal. 24, 1496-1503, (2012).
40. Zhang, L., Malik, S., Kelley, G. G., Kapiloff, M. S. & Smrcka, A. V. Phospholipase C epsilon scaffolds to muscle-specific A kinase anchoring protein (mAKAPbeta) and integrates multiple hypertrophic stimuli in cardiac myocytes. J. Biol. Chem. 286, 23012-23021, (2011).
41. Marx, S. O. et al. Phosphorylation-dependent regulation of ryanodine receptors: a novel role for leucine/isoleucine zippers. J. Cell Biol. 153, 699-708, (2001).
42. Li, J. et al. The mAKAPbeta scaffold regulates cardiac myocyte hypertrophy via recruitment of activated calcineurin. J. Mol. Cell. Cardiol. 48, 387-394, (2010).
43. Passariello, C. L. et al. p90 ribosomal S6 kinase 3 contributes to cardiac insufficiency in alpha-tropomyosin Glu180Gly transgenic mice. Am J Physiol Heart Circ Physiol 305, H1010-1019, (2013).

1. A method for treating damage to or degenerative diseases of the nervous system, including neurons and glial cells in the brain, spinal cord and visual system including the retina and optic nerves, applied to nervous system cells after trauma, or in neurodegenerative diseases including without limitation glaucoma, traumatic optic neuropathy, ischemic optic neuropathy, retinal or macular degeneration whether age-related or inherited, Alzheimer's disease, stroke, in a mammal, the method comprising:
administering an effective dose of a PDE4D3 displacing agent to the affected nerve.

2. The method of claim 1 wherein the method comprises treatment of optic neuropathy.

3. The method of claim 2, wherein the optic neuropathy affects retinal ganglion cells (RGC).

4. The method of any of claim 2, wherein the optic neuropathy is one of glaucoma, ischemic optic neuropathy, traumatic optic neuropathy, optic nerve drusen and optic neuritis.

5. The method of claim 1, wherein the PDE4D3 displacing agent corresponds to an N-terminal peptide of human PDE4D3.

6. The method of claim 5, wherein the PDE4D3 displacing agent comprises the amino acid sequence (SEQ ID NO:1) MMHVNNFPFRRHXWICFDVD, where X is any amino acid.

7. The method of claim 6, wherein X is E.

8. The method of claim 6, wherein an isolated peptide of SEQ ID NO:1 is fused to a polypeptide sequence other than PDE4D3.

9. The method of claim 8, wherein the polypeptide sequence other than PDE4D3 is a transporter domain.

10. The method of claim 5, wherein the PDE4D3 displacing agent comprises a genetic vector encoding amino acid sequence (SEQ ID NO:1) MMHVNNFPFRRHXWICFDVD, where X is any amino acid, operably linked to a promoter active in nerve cells.

11. The method of claim 10, wherein X is E.

12. The method of claim 11, wherein the genetic vector is a viral vector.

13. The method of claim 12, wherein the viral vector is an adeno-associated virus.

14. The method of claim 1, wherein the PDE4D3 displacing agent is administered to the eye.

15. The method of claim 14, wherein the PDE4D3 displacing agent is administered by eye drops, intravitreal injection, subconjunctival, or periocular route of administration.

16. The method of claim 1, wherein the PDE4D3 displacing agent is administered in combination with activation or administration of a neurotrophic factor or visual or electrical stimulation, where the activity of the neurotrophic factor or visual or electrical stimulation is potentiated by administration of the PDE4D3 displacing agent.

17. The method of claim 16, wherein the neurotrophic factor is selected from brain-derived neurotrophic factor (BDNF), ciliary neurotrophic factor (CNTF), glial cell line-derived neurotrophic factor (GDNF), neurotrophin-4, and sciatic nerve (ScN)-derived factor.

18. The method of claim 1, wherein PDE4D3 displacing agent is administered in combination with an anti-glaucoma medicament.

19. A PDE4D3 displacing agent, optionally for use in the method of claim 1.

20. A therapeutic composition comprising the PDE4D3 displacing agent of claim 19, and a pharmaceutically acceptable excipient.

21. The PDE4D3 displacing agent of claim 19, corresponding to an N-terminal peptide of human PDE4D3.

22. The PDE4D3 displacing agent of claim 21, comprising the amino acid sequence (SEQ ID NO:1) MMHVNNFPFRRHXWICFDVD, where X is any amino acid.

23. The agent of claim 22, wherein X is E.

24. The agent of claim 22, wherein an isolated peptide of SEQ ID NO:1 is fused to a polypeptide sequence other than PDE4D3.

25. The agent of claim 24, wherein the polypeptide sequence other than PDE4D3 is a transporter domain.

26. The agent of claim 21, wherein the PDE4D3 displacing agent comprises a genetic vector encoding amino acid sequence (SEC) ID NO:1) MMHVNNFPFRRHXWICFDVD, where X is any amino acid, operably linked to a promoter active in an optic nerve.

27. The agent of claim 26, wherein X is E.

28. The agent of claim 27, wherein the genetic vector is a viral vector.

29. The agent of claim 28, wherein the viral vector is an adeno-associated virus.
